(19) United States
(12) Patent Application Publication
(10) Pub. No.: US 2025/0009891 A1
(43) Pub. Date: Jan. 9, 2025

(54) CONJUGATES OF MONOCYCLIC BETA-LACTAMS AND SIDEROPHORE MIMETICS

(71) Applicant: UNIVERSITY OF NOTRE DAME DU LAC, SOUTH BEND, IN (US)

(72) Inventors: Marvin MILLER, SOUTH BEND, IN (US); Rui LIU, SOUTH BEND, IN (US); Patricia MILLER, SOUTH BEND, IN (US)

(21) Appl. No.: 18/685,157

(22) PCT Filed: Aug. 22, 2022
(86) PCT No.: PCT/US2022/041051

§ 371 Date: Feb. 20, 2024

Related U.S. Application Data
(60) Provisional application No. 63/235,536, filed on Aug. 20, 2021.

(51) Int. Cl. A61K 47/55; A61K 45/06; A61K 47/54; A61P 31/04

(52) U.S. Cl. CPC A61K 47/552; A61K 45/06; A61K 47/545; A61P 31/04

(57) ABSTRACT

Monocyclic beta-lactams with antibiotic activity were synthesized over 40 years ago. Extensive early structure-activity relationship (SAR) studies, especially in the 1980s, emphasized the need for heteroatom activation of monocyclic beta-lactams and led to studies of oxamazins, monobactams, monosulfactams and monocarbams with various side chains and peripheral substitution that revealed potent activity against select strains of Gram-negative bacteria. Aztreonam, still the only clinically used monobactam, has notable activity against many Gram-negative bacteria but limited activity against some of the most problematic multi-drug resistant (MDR) strains of Pseudomonas aeruginosa and Acinetobacter baumannii. This invention reports that extension of the side chain of monocyclic beta-lactams, notably aztreonam, is tolerated and especially that coupling of the side chain free acid with a bis-catechol siderophore mimetic significantly improves activity against MDR strains of Gram-negative bacteria, including multidrug resistant and beta-lactamase producing strains of most concern.

### CROSS REFERENCE TO THE RELATED APPLICATION

This application claims priority to U.S. Provisional Application No. 63/235,536, filed Aug. 20, 2021, which is hereby incorporated by reference in its entirety.

### REFERENCE TO GOVERNMENT RIGHTS

This invention was made with government support under Grant No. R37 AI054193 awarded by the National Institutes of Health. The government has certain rights in the invention.

### FIELD

This invention describes the design, syntheses and antibacterial studies of novel conjugates of monocyclic β-lactams and siderophores. The compounds show enhanced antibacterial activity against Gram-negative bacteria, including multidrug resistant and β-lactamase producing strains of most concern.

### BACKGROUND OF THE INVENTION

As the reactive warhead in classical penicillin, cephalosporin, carbapenem and related antibiotics, the β-lactam core is referred to as the “enchanted ring”. These antibiotics positively influenced life and have been attributed to a significant increase in life expectancy over the past century. As shown in FIG. 1, β-lactam antibiotics are fused bicyclic compounds with pendant functionality that is necessary for recognition as they induce bacterial cell wall disruption. The bicyclic ring strain enhances the inherent reactivity of the β-lactam ring towards nucleophilic ring opening while interfering with bacterial cell wall synthesis. The extensive beneficial use and overuse of these important antibiotics has promoted the development of resistance to each successive generation of bicyclic β-lactams with concomitant loss of efficacy primarily because of extensive proliferation of β-lactamases that destroy the β-lactams before they reach their target. Health agencies have warned that over the next few decades loss of antibiotic efficacy will result in millions of deaths with added economic burdens of multi trillions of dollars. The WHO has identified several multidrug and even totally drug resistant strains of bacteria that are of particular concern including β-lactamase producing strains of Acinetobacter baumannii and Pseudomonas aeruginosa. Although thousands of derivatives of bicyclic β-lactams have been prepared, most have relied on fermentation processes to provide the bicyclic framework for subsequent peripheral modification.

To date, no practical totally chemical syntheses of the penicillins or cephalosporins are available. In contrast, more than 40 years ago, a hydroxamate-mediated N-C₄ biomimetic cyclization process (5 to 6, Scheme 1) was developed to make it possible to efficiently synthesize the β-lactam core from β-hydroxy carboxylic acids (4) with complete control of peripheral functionality and stereochemistry. The same process also introduced the concept of heteroatom activation rather than just bicyclic activation of the enchanted ring as manifested by the subsequent rapid disclosure of the oxamazins (7), monosulfactams (8), monobactams (9), monocarbams (10) and other moncyclic β-lactams. Development of this chemistry also coincided with the discovery of natural N-sulfated β-lactams (monobactams).

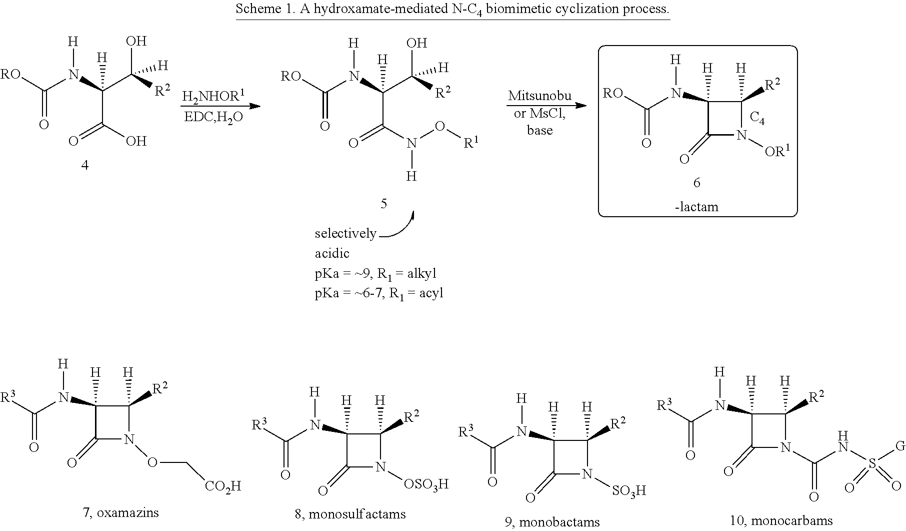

Although the natural monobactams were not highly active antibiotics, use of the N-C₄ cyclization chemistry allowed syntheses of very active anti-Gram-negative bacteria monocyclic β-lactam antibiotics. Extensive structure-activity-relationship (SAR) studies resulted in peripheral optimization (FIG. 2) that led to the first, and still only marketed monobactam, aztreonam (9a). Many other very active monobactams, including carumonam (11) and LYS228 (12) have been reported, but are not in clinical use, mainly because of marketing limitations. Aztreonam is derived from the natural, readily available amino acid, L-threonine by modifications of the N-C₄ cyclization chemistry. It was approved by the FDA in 1986 and is still used as an injectable antibiotic to treat infections caused by Gram-negative bacteria including some that produce β-lactamases. However, aztreonam lacks efficacy against some of the MDR Gram-negative bacterial strains of greatest concern. Therefore, there is unmet need to develop new compounds with improved efficacy against the MDR Gram-negative bacterial stains. To address the need for improvement of the antibiotic activity of monobactams against MDR Gram-negative bacteria, provided herein is an invention directed to conjugates (27 and 30) of simple bis catechol siderophore mimetics and aztreonam (9a) that have enhanced and potent activity against Gram-negative bacteria, including those resistant to aztreonam itself.

The design, syntheses and studies of siderophore-antibiotic conjugates that mimic natural sideromycins utilize essential iron sequestration processes to actively transport antibiotics into targeted pathogenic strains of bacteria. Most active synthetic sideromycins incorporate β-lactams as the antibiotic (“warhead”) component. In many cases, siderophore conjugates of penicillins (13, FIG. 3) and cephalosporins (14, 15) have enhanced activity due to active transport and circumvention of efflux, but some are still susceptible to deactivation by β-lactamases. However, cefiderocol (16), now also called Fetroja based on the iron transport mediated Trojan Horse concept, is stable to most β-lactamases and has received FDA approval. β-lactamases can be exploited to release antibiotics from synthetic sideromycins. For example, a synthetic siderophore-cephalosporin-oxazolidinone conjugate (17) was potently active against cephalosporinase producing strains of A. baumannii. By using the siderophore to actively transport the conjugate into the targeted bacteria, the β-lactamase destroyed the cephalosporin and released the normally Gram-positive antibiotic intracellularly and allowed it to kill Gram-negative bacteria. While effective, this dual drug conjugate requires extensive synthesis.

To avoid detrimental β-lactamase problems associated with conjugates of classical penicillins and cephalosporins, siderophore conjugates of non-β-lactam antibiotics have been studied, including large complex compounds like daptomcyin and teicoplanin. In vitro and in vivo studies demonstrated the effectiveness of the sideromycin approach to repurposing normally Gram-positive only, antibiotics to target Gram-negative bacteria. The reduced susceptibility of monobactams to β-lactamases also prompted us to further consider this class of small, structurally more simple compounds for siderophore conjugation. The primary concern was that more extensive modification of the monobactam periphery might negatively affect the previously established SAR (9a, FIG. 2). However, decades ago iron binding hydroxypyridone substituted monocarbams such as U-78,608 (18) and pirazmonam (SQ-83,360, 19), as well as catechol SQ-83,280 (20), were reported to have activity against Gram-negative bacteria even though they incorporated extensive modification beyond the original monobactam SAR suggestions (FIG. 4). Synthesis and studies of MC-1 (21) a hydroxypyridone containing monocarbam with diol substitution indicated that it was hydrolytically stable and that it exhibited potent Gram-negative antibacterial activity. The monosulfactam BAL 30072 (22) and related compounds also replaced the usual aminothiazooxime carboxylic acid with a substituted hydroxypyridone. While hydroxypyridones are not common natural siderophore iron binding ligands, they mimic catechols and their monocyclic β-lactam derivatives and utilize siderophore transport to promote activity against Gram-negative bacteria. Because of early marketing limitations and other factors at the time, none of these compounds were used clinically. Their activity revealed that more extensive peripheral modification of the monocyclic β-lactam core was tolerated. The known syntheses of C₄-substituted monobactams are lengthy since the corresponding β-hydroxy-α-amino acid precursors (4, with functionalized R²) are not readily available and require alternative syntheses. Therefore, there is unmet need to develop a simple alternative synthesis route. Provided herein is an invention directed to simple synthesis of a C₄-substituted monobactam with a bis-catechol siderophore mimetic. This conjugate (23) was extremely active against problematic Gram-negative bacteria, including carbapenemase and cephalosporinase producing strains of P. aeruginosa and A. baumannii, whereas aztreonam was not active.

While there has been skepticism about the clinical potential of siderophore antibiotic conjugates (sideromycins), the potential of this so-called Trojan Horse approach to enhancing activity and even repurposing normally Gram-positive antibiotics and other agents merits continued attention. The recent FDA approval of cefiderocol (FeTroja, 16) emphasizes the value of this approach. It should also be recalled that natural sideromycins, including albomycin, were successfully used in the clinic to treat antibiotic resistant infections even in the 1950s. Appropriately designed synthetic sideromycins have the potential to allow much needed development of both broad spectrum and narrowly targeted antibiotics.

### SUMMARY OF THE INVENTION

Provided herein is an invention directed to a compound of Formula (I):

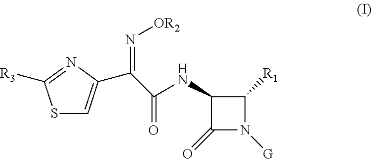

or a pharmaceutically acceptable salt or zwitterion thereof, wherein
G is —OR^{A}, —OCH₂C(═O)X, OCHRC(═O)X, —C(═O)X, —S(═O)₂X, —OS(═O)₂X, —C(═O)NH—S(═O)₂G′, P(═O)X or —P(═O)X,
G′ is OR^{A}, —OCH₂C(═O)X, OCHRC(═O)X, —C(═O)X, —S(═O)₂X, —OS(═O)₂X, P(═O)X or —P(═O)X,
X is H, —OR^{A} or —N(R^{A})₂,
R₁ is H, or —(C₁-C₁₂)alkyl optionally substituted by X or Z,
Z is R^{A}, OR^{A}, —SR^{A}, —C(═O)N(R^{A})₂, or —N(R^{A})₂,
R₂ is —CH₂C(═O)W, —CHR⁴C(═O)W, —C(R⁴)₂C(═O)W, —C(R⁴)₂C(═O)NHOCH₂C(═O)W, —C(R⁴)₂C(═O)NHOCH₂C(═O)OH, —C(R⁴)₂C(═O)NHOC(R⁴)₂C(═O)OH, or —C(R⁴)₂C(═O)NHOC(R⁴)₂C(═O)W,
R₃ is H, Z, or R⁴,
each R⁴ is independently H, Z, —(C₁-C₁₂)alkyl, —(C₃-C₈)cycloalkyl, —(C₃-C₆)heterocycloalkyl, or —(C₁-C₈)N(R^{A})R^{B}, or two R⁴ are taken together with the carbon atom to which both are attached to form a —(C₃-C₈)cycloalkyl or —(C₃-C₆)heterocycloalkyl,
each R^{A} is independently H, —(C₁-C₁₂)alkyl, —(C₃-C₈)cycloalkyl, phenyl, aryl, or heteroaryl,
R^{B} is H, —(C₁-C₁₂)alkyl, —(C₃-C₈)cycloalkyl, or phenyl,
W is Sid or linker-Sid,
Sid is a siderophore moiety,
linker is —NH-Q-C(═O)O—, —NH—C(═O)-Q-C(═O)O—, —C(═O)-Q-, —C(═O)O-Q-, —C(═O)-Q-C(═O)—NH—, —O-Q-O—, —O-Q-, —NH-Q-, —NH-Q-NH—, —NH-Q-O—, or the combination thereof,
Q is a —(C₁-C₁₂)alkylene optionally substituted by one or more selected from the group consisting of —OH, —COOH, —(C₁-C₁₂)alkyl, —(C₁-C₁₂)alkenyl, —(C₁-C₁₂)alkylene —OH, —(C₁-C₁₂)alkylene —NH₂, —NH—(C₁-C₁₂)alkylene-COOH, —NH—C(═O)—(C₁-C₁₂)alkylene —COOH, —C(═O)—(C₁-C₁₂)alkylene —C(═O)—NH₂, —O—(C₁-C₁₂)alkylene-OH, —NH—(C₁-C₁₂)alkylene —NH₂, and —NH—(C₁-C₁₂)alkylene —OH.

Also provided herein is a method of treating a bacterial infection in a subject in need thereof, comprising administering to the said subject a therapeutically effective amount of the compound of Formula (I) as described above or a pharmaceutically acceptable salt or zwitterion thereof or a therapeutically effective amount of the pharmaceutical composition comprising the compound of Formula (I) as described above.

Further provided herein is a process of preparing a compound of Formula (I), or a pharmaceutically acceptable salt or zwitterion thereof, the process comprising: contacting a compound of Formula (I′-1)

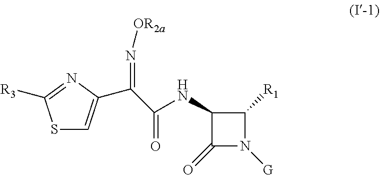

with a compound of Formula (II′-1) or Formula (II′-2)

R₈—U₁—NH₂, (II′-1), or

R₈—U₁-L′—NH₂ (II′-2)

under suitable conditions to produce the compound having Formula (I)

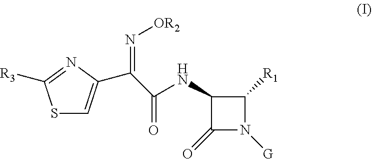

wherein
G is —OR^{A}, —OCH₂C(═O)X, OCHRC(═O)X, —C(═O)X, —S(═O)₂X, —OS(═O)₂X, —C(═O)NH—S(═O)₂G′, P(═O)X or —P(═O)X,
G′ is OR^{A}, —OCH₂C(═O)X, OCHRC(═O)X, —C(═O)X, —S(═O)₂X, —OS(═O)₂X, P(═O)X or —P(═O)X,
X is H, —OR^{A} or —N(R^{A})₂,
R₁ is H, or —(C₁-C₁₂)alkyl optionally substituted by X or Z,
Z is R^{A}, —OR^{A}, —SR^{A}, —C(═O)N(R^{A})₂, or —N(R^{A})₂,
R₂ₐ is —CH₂C(═O)OH, —CHR⁴C(═O)OH, —C(R⁴)₂C(═O)OH, or —C(R⁴)₂C(═O)NHOCH₂C(═O)OH, —C(R⁴)₂C(═O)NHOC(R⁴)₂C(═O)OH,
R₂ is —CH₂C(═O)W′, —CHR⁴C(═O)W′, —C(R⁴)₂C(═O)W′, —C(R⁴)₂C(═O)NHOCH₂C(═O)W′, —C(R⁴)₂C(═O)NHOCH₂C(═O)OH, —C(R⁴)₂C(═O)NHOC(R⁴)₂C(═O)OH, or —C(R⁴)₂C(═O)NHOC(R⁴)₂C(═O)W′,
R₃ is H, Z, or R⁴,
each R⁴ is independently H, Z, —(C₁-C₁₂)alkyl, —(C₃-C₈)cycloalkyl, —(C₃-C₆)heterocycloalkyl, or —(C₁-C₈)N(R^{A})R^{B}, or two R⁴ are taken together with the carbon atom to which both are attached to form a —(C₃-C₈)cycloalkyl or —(C₃-C₆)heterocycloalkyl,
each R^{A} is independently H, —(C₁-C₁₂)alkyl, —(C₃-C₈)cycloalkyl, phenyl, aryl, or heteroaryl,
R^{B} is H, —(C₁-C₁₂)alkyl, —(C₃-C₈)cycloalkyl, or phenyl,
W′ is —NH-U₁-R₈ or —NH-L′—U₁-R₈,
L′ is -Q-C(═O)O—, —C(═O)-Q-C(═O)O—, —C(═O)-Q-, —C(═O)O-Q-, —C(═O)-Q-C(═O)—, —O-Q-, -Q-, -Q-NH—, -Q-O—, or the combination thereof,
each Q is independently a —(C₁-C₁₂)alkylene optionally substituted by one or more selected from the group consisting of —OH, —COOH, ═O, —NH₂, —(C₁-C₁₂)alkyl, —(C₁-C₁₂)alkenyl, —(C₁-C₁₂)alkylene —OH, —(C₁-C₁₂)alkylene —NH₂, —NH—(C₁-C₁₂)alkylene-COOH, —NH—C(═O)—(C₁-C₁₂)alkylene —COOH, —C(═O)—(C₁-C₁₂)alkylene —C(═O)—NH₂, —O—(C₁-C₁₂)alkylene-OH, —NH—(C₁-C₁₂)alkylene —NH₂, and —NH—(C₁-C₁₂)alkylene —OH.
U₁ is a covalent bond, or optionally substituted -Q-, —C(═O)—, —O—C(═O)—, —CHR₈—, —CR₁₀R₈—, —C(═O)—NH—, —C(═O)—NR₈—, —C(═O)—N(OH)—, —NH—, —NR₈—, —NR₁₀—, —NH—CHR₈—C(═O)—, —R₁₁—, or the combination thereof,
each R₈ is each independently H, —OH, —COOH, —NH₂, -Q-OH, —V, -Q-V, —NH—C(═O)—V, -Q-NH—C(═O)—V, —NR₁₀—C(═O)—V, —NR₁₀—V, —C(═O)—V, —O—C(═O)—V, —C(═O)—R₁₁—V, —NH—C(═O)—R₁₁—V, —C(═O)—R₁₁-aryl, or linker-H,
R₁₀ is —NH—C(O)-Q-COOH, —C(O)-Q-COOH, -Q-COOH, or —COOH,
R₁₁ is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S, V═—N(OH)—C(═O)—NH₂, —N(OH)—C(═O)H, N(OH)—C(═O)-linker, —N(OH)—C(═O)—(C₁-C₁₂)alkyl, —N(OH)—C(═O)—(C₁-C₁₂)alkenyl, —N(OH)—C(═O)-Q-OH, —N(OH)—C(═O)-Q-COOH,

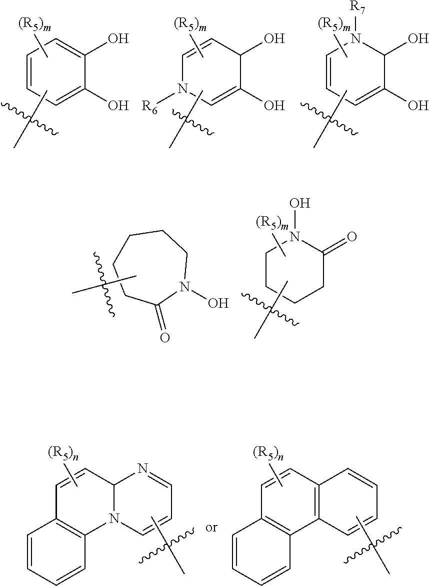

m is an integral selected from 0-3,
n is an integral selected from 0-10,
each R₅ is independently —(C₁-C₁₂)alkyl, halogen, —OH, —COOH, —NH₂, -linker-H, or —C(═O)-linker-H, and
R₆ and R₇ are independently H, —COOH, or —OR^{A}.

### BRIEF DESCRIPTION OF THE DRAWINGS

FIG. 1 descries classical bicyclic β-lactam antibiotics.

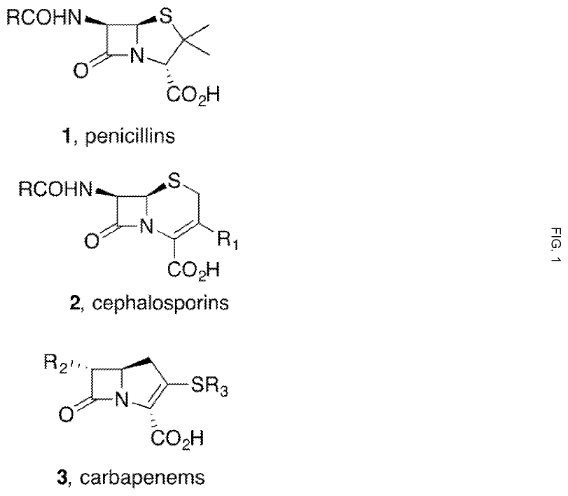

FIG. 2 provides a SAR overview of aztreonam (9a) and structures of related monobactams.

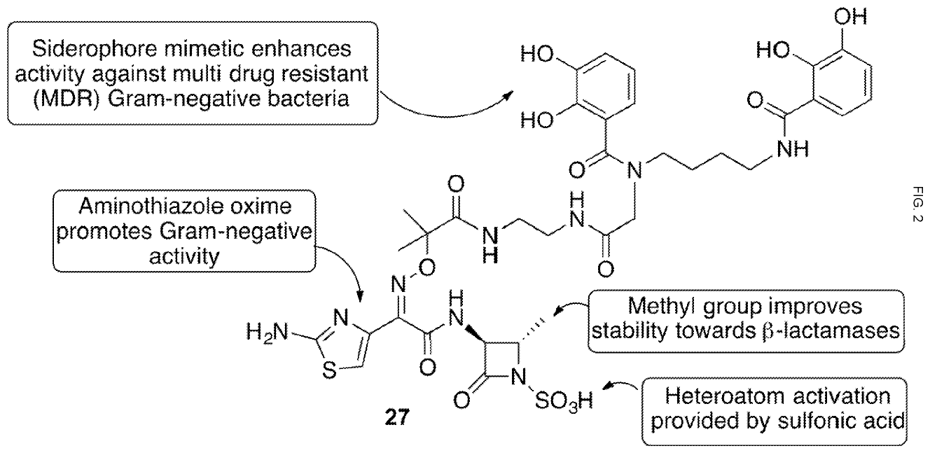

FIG. 3 provides representative known bis-catechol antibiotic conjugates.

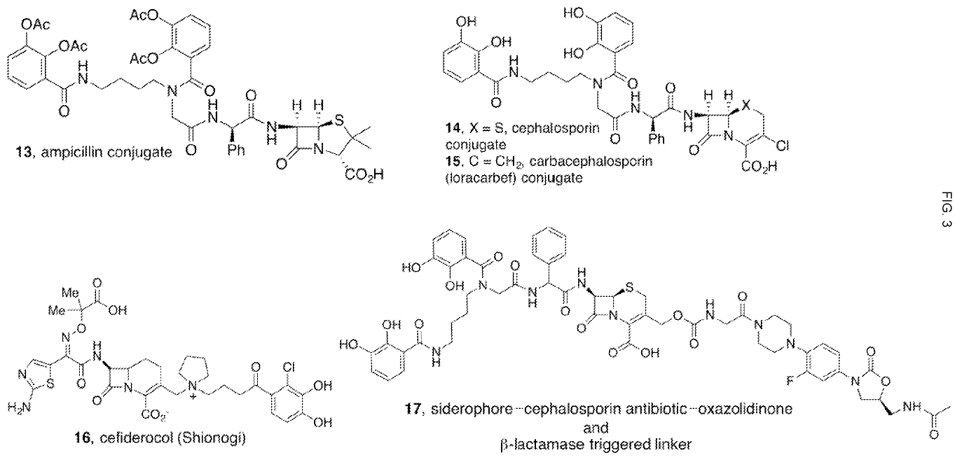

FIG. 4 describes monocarbams, monosulfactam and monobactam conjugates.

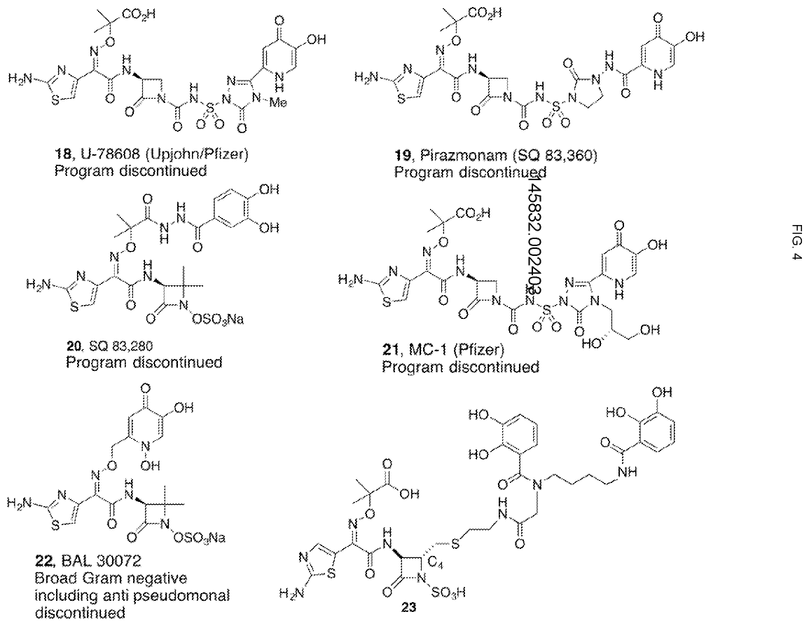

FIG. 5a provides representative siderophores, analogs and mimetics of amino acid based hydroxamic acids.

FIG. 5b provides representative siderophores, analogs and mimetics of amino alkane based hydroxamic acids.

FIG. 5c provides representative siderophores, analogs and mimetics of catechols and hydroxyl pyridines.

FIG. 5d provides representative siderophores, analogs and mimetics of mixed ligand siderophores and mimetics

### DETAILED DESCRIPTION OF THE INVENTION

Unless defined otherwise, all technical and scientific terms have the same meaning as is commonly understood by one of ordinary skill in the art to which the embodiments disclosed belongs. In the event that there is a plurality of definitions for terms cited herein, those in this section prevail unless otherwise stated. All patents, applications, published applications, and other publications cited herein are incorporated by reference in their entirety.

As used herein, the terms “a” or “an” means that “at least one” or “one or more” unless the context clearly indicates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments.

As used herein, the term “additive” or “coupling additive” means a reagent that is suitable in combination with a coupling reagent in coupling reactions to inhibit side reactions and reduce or eliminate racemization. In some embodiments, an additive is, but not limited to, ethyl cyanohydroxyiminoacetate, N-hydroxysuccinimide (HOSu), N-hydroxy-5-norbornene2,3-dicarboximide (HONB), 1-hydroxybenzotriazole (HOBt), 6-chloro-1-hydroxybenzotriazole (6-Cl-HOBt), 1-hydroxy-7-azabenzotriazole (HOAt) or 3-hydroxy-4-oxo-3,4-dihydro-1,2,3-benzotriazine (HODhbt), aza derivative of 3-hydroxy-4-oxo-3,4-dihydro-1,2,3-benzotriazine (HODhat), 4-(N,N-Dimethylamino)pyridine) (DMAP), N-hydroxysuccinimide (HOSu), N-hydroxy-5-norbornene-2,3-dicarboximide (HONB), or any combinations thereof.

As used herein, the term “alcohol” means any organic compound in which a hydroxyl group (—OH) is bound to a carbon atom, which in turn is bound to other hydrogen and/or carbon atoms. For example, the term “alcohol” means a straight or branched alkyl-OH group of 1 to 20 carbon atoms, including, but not limited to, methanol, ethanol, n-propanol, isopropanol, t-butanol, and the like. In some embodiments, the alkyl-OH chain is from 1 to 10 carbon atoms in length, from 1 to 8 carbon atoms in length, from 1 to 6 carbon atoms in length, from 1 to 4 carbon atoms in length, from 2 to 10 carbon atoms in length, from 2 to 8 carbon atoms in length, from 2 to 6 carbon atoms in length, or from 2 to 4 carbon atoms in length.

As used herein, the terms “alkoxy”, “phenyloxy”, “benzoxy” and “pyrimidinyloxy” refer to an alkyl group, phenyl group, benzyl group, or pyrimidinyl group, respectively, each optionally substituted, that is bonded through an oxygen atom. For example, the term “alkoxy” means a straight or branched —O-alkyl group of 1 to 20 carbon atoms, including, but not limited to, methoxy, ethoxy, n-propoxy, isopropoxy, t-butoxy, and the like. In some embodiments, the alkoxy chain is from 1 to 10 carbon atoms in length, from 1 to 8 carbon atoms in length, from 1 to 6 carbon atoms in length, from 1 to 4 carbon atoms in length, from 2 to 10 carbon atoms in length, from 2 to 8 carbon atoms in length, from 2 to 6 carbon atoms in length, or from 2 to 4 carbon atoms in length.

As used herein, the term “alkyl” means a saturated hydrocarbon group which is straight-chained or branched. An alkyl group can contain from 1 to 20, from 2 to 20, from 1 to 10, from 2 to 10, from 1 to 8, from 2 to 8, from 1 to 6, from 2 to 6, from 1 to 4, from 2 to 4, from 1 to 3, or 2 or 3 carbon atoms. Examples of alkyl groups include, but are not limited to, methyl (Me), ethyl (Et), propyl (e.g., n-propyl and isopropyl), butyl (e.g., n-butyl, t-butyl, isobutyl), pentyl (e.g., n-pentyl, isopentyl, neopentyl), hexyl, isohexyl, heptyl, 4,4-dimethylpentyl, octyl, 2,2,4-trimethylpentyl, nonyl, decyl, undecyl, dodecyl, 2-methyl-1-propyl, 2-methyl-2-propyl, 2-methyl-1-butyl, 3-methyl-1-butyl, 2-methyl-3-butyl, 2-methyl-1-pentyl, 2,2-dimethyl-1-propyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, and the like.

As used herein, the term “alkylene” or “alkylenyl” means a divalent alkyl linking group. An example of an alkylene (or alkylenyl) is methylene or methylenyl (—CH₂—).

As used herein, the term “alkynyl” means a straight or branched alkyl group having one or more triple carbon-carbon bonds and 2-20 carbon atoms, including, but not limited to, acetylene, 1-propylene, 2-propylene, and the like. In some embodiments, the alkynyl chain is 2 to 10 carbon atoms in length, from 2 to 8 carbon atoms in length, from 2 to 6 carbon atoms in length, or from 2 to 4 carbon atoms in length.

As used herein, the terms “ambient temperature” and “room temperature” or “RT”, as used herein, are understood in the art, and refer generally to a temperature, e.g. a reaction temperature, that is about the temperature of the room in which the reaction is carried out, for example, a temperature from about 20° C. to about 30° C., such as at or about 25° C.

As used herein, the term “amide” means to a functional group containing a carbonyl group linked to a nitrogen atom or any compound containing the amide functional group. For example, amides are derived from carboxylic acid and an amine.

As used herein, the term “aryl” means a monocyclic, bicyclic, or polycyclic (e.g., having 2, 3 or 4 fused rings) aromatic hydrocarbons. In some embodiments, aryl groups have from 6 to 20 carbon atoms or from 6 to 10 carbon atoms. Examples of aryl groups include, but are not limited to, phenyl, naphthyl, anthracenyl, phenanthrenyl, indanyl, indenyl, tetrahydronaphthyl, and the like. Examples of aryl groups include, but are not limited to:

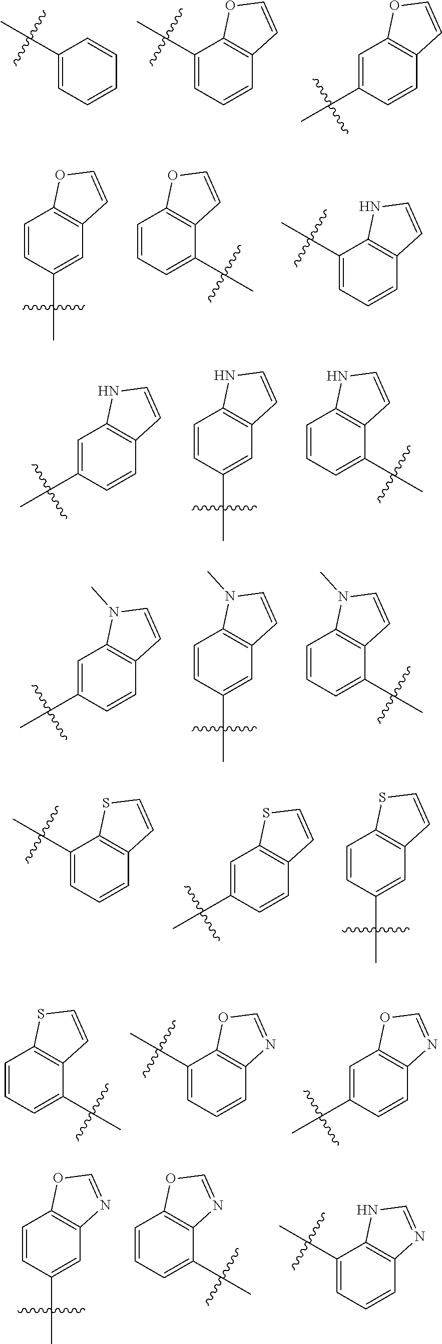

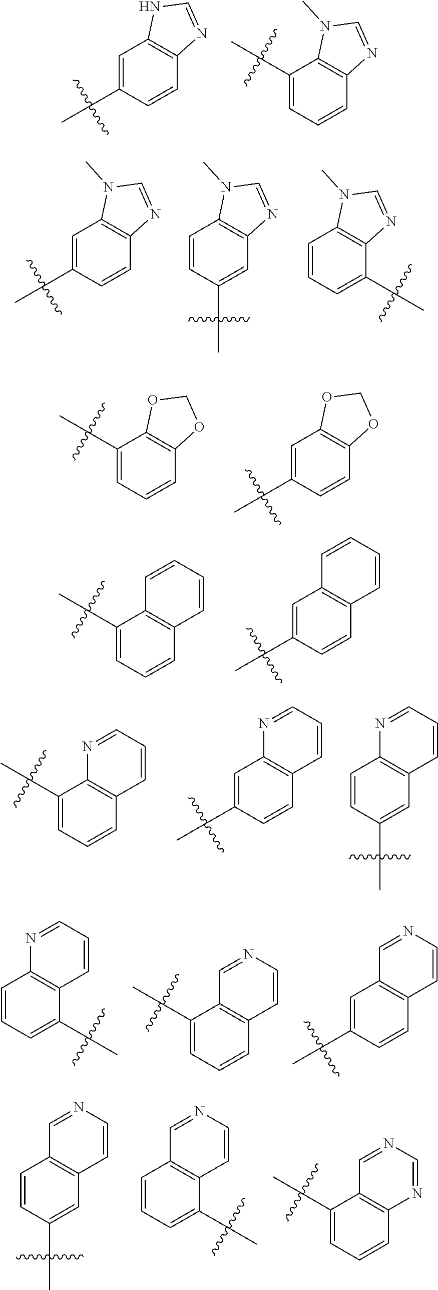

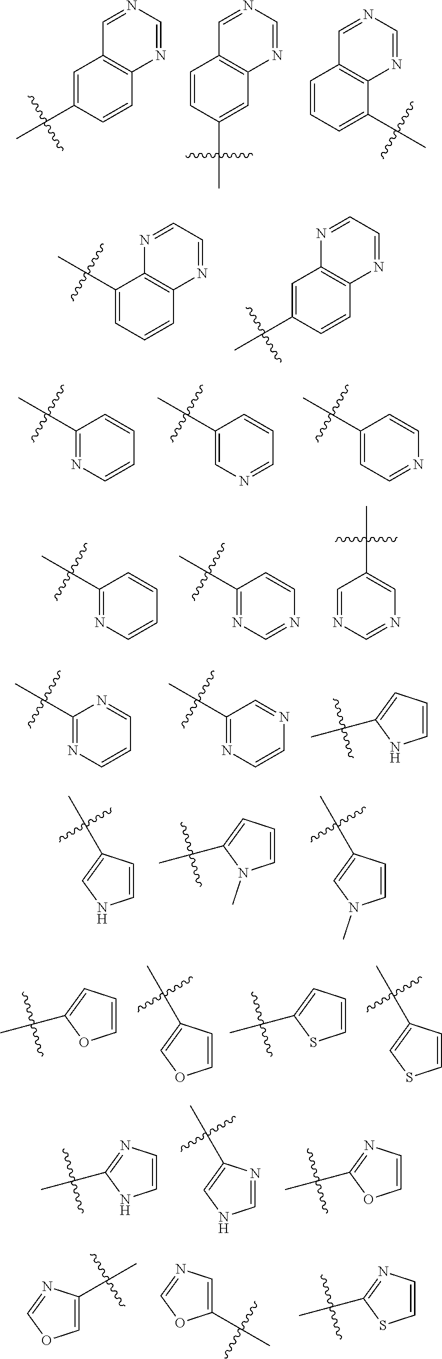

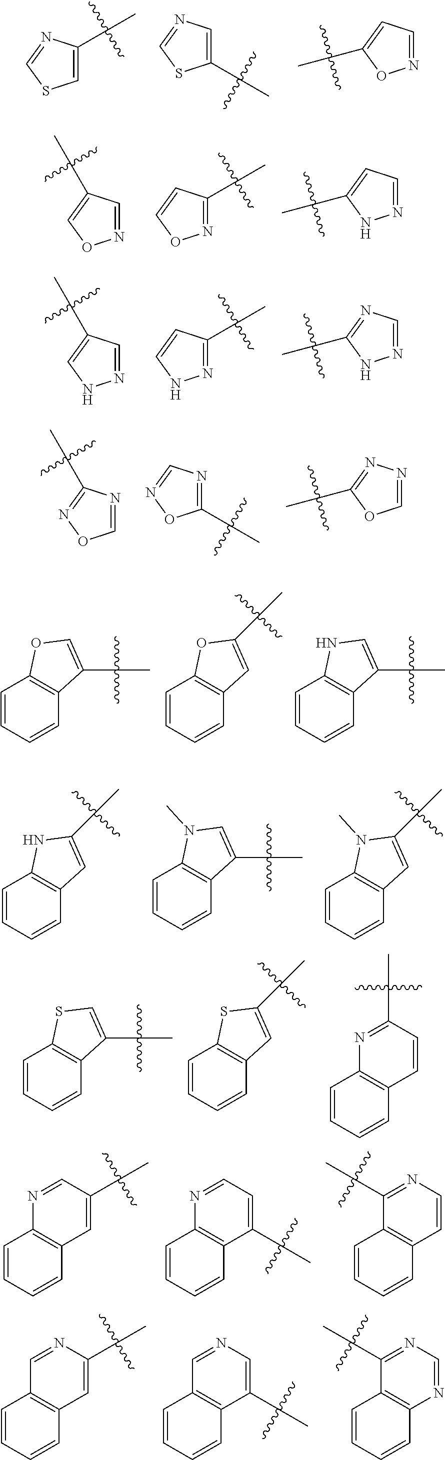

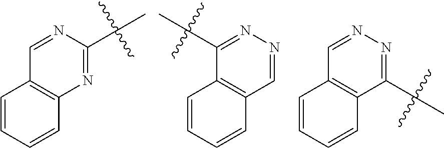

As used herein, the term “carbocycle” means a 5-6, or 7-membered, saturated or unsaturated cyclic ring, optionally containing, S, or N atoms as part of the ring. Examples of carbocycles include, but are not limited to, cyclopentyl, cyclohexyl, cyclopenta-1,3-diene, phenyl, and any of the heterocycles recited above.

As used herein, the term, “compound” means all stereoisomers, tautomers, and isotopes of the compounds described herein.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the term “contacting” or “mixing” or “adding” means bringing together of two compounds/atoms to form at least one covalent bond between the compounds or atoms.

As used herein, the term “coupling reagent” or “peptide coupling reagent” means a reagent that facilitate to form an amide bond between an amine and carboxylic acid including but not limited to carbodiimides, aminium/uronium and phosphonium salts, and propanephosphonic acid anhydride. For example, the coupling reagent is diisopropylcarbodiimide (DIC), dicyclohexylcarbodiimide (DCC), 1-Ethyl-3-(3-dimethylaminopropyl)carbodiimide (EDC, EDAC or EDCI), 1-[Bis(dimethylamino)methylene]-1H-1,2,3-triazolo[4,5-b]pyridinium 3-oxide hexafluorophosphate, Hexafluorophosphate Azabenzotriazole Tetramethyl Uronium (HATU), 2-(1H-benzotriazol-1-yl)-1,1,3,3-tetramethyluronium hexafluorophosphate, Hexafluorophosphate Benzotriazole Tetramethyl Uronium (HBTU), O-(1H-6-Chlorobenzotriazole-1-yl)-1,1,3,3-tetramethyluronium hexafluorophosphatO-(1H-6-Chlorobenzotriazole-1-yl)-1,1,3,3-tetramethyluronium hexafluorophosphate (HCTU), Benzotriazol-1-yloxy)tripyrrolidinophosphonium hexafluorophosphate (PyBOP), 7-Azabenzotriazol-1-yloxy)tripyrrolidinophosphonium hexafluorophosphate (PyAOP), Propanephosphonic acid anhydride (PPAA, T3P), or any combination thereof.

As used herein, the term “cyano” means —CN.

As used herein, the term “cycloalkyl” means non-aromatic cyclic hydrocarbons including cyclized alkyl, alkenyl, and alkynyl groups that contain up to 20 ring-forming carbon atoms.

Cycloalkyl groups can include mono- or polycyclic ring systems such as fused ring systems, bridged ring systems, and spiro ring systems. In some embodiments, polycyclic ring systems include 2, 3, or 4 fused rings. A cycloalkyl group can contain from 3 to 15, from 3 to 10, from 3 to 8, from 3 to 6, from 4 to 6, from 3 to 5, or 5 or 6 ring-forming carbon atoms. Ring-forming carbon atoms of a cycloalkyl group can be optionally substituted by oxo or sulfido. Examples of cycloalkyl groups include, but are not limited to, cyclopropyl, cyclobutyl, cyclopentyl, cyclohexyl, cycloheptyl, cyclooctyl, cyclononyl, cyclopentenyl, cyclohexenyl, cyclohexadienyl, cycloheptatrienyl, norbornyl, norpinyl, norcarnyl, adamantyl, and the like. Also included in the definition of cycloalkyl are moieties that have one or more aromatic rings fused (having a bond in common with) to the cycloalkyl ring, for example, benzo or thienyl derivatives of pentane, pentene, hexane, and the like (e.g., 2,3-dihydro-1H-indene-1-yl, or 1H-inden-2(3H)-one-1-yl).

As used herein, the term “cycloheteroalkyl” means as used herein alone or as part of another group refers to a 5-, 6- or 7-membered saturated or partially unsaturated ring which includes 1 to 2 hetero atoms such as nitrogen, oxygen and/or sulfur, linked through a carbon atom or a heteroatom, where possible, optionally via the linker (CH₂)n (where n is 0, 1, 2 or 3). The above groups may include 1 to 4 substituents such as alkyl, halo, oxo and/or any of the substituents for alkyl or aryl set out herein. In addition, any of the cycloheteroalkyl rings can be fused to a cycloalkyl, aryl, heteroaryl or cycloheteroalkyl ring.

As used herein, the terms “for example” and “such as,” and grammatical equivalences thereof.

As used herein, the term “halo” means halogen groups including, but not limited to fluoro, chloro, bromo, and iodo.

As used herein, the term “haloalkoxy” means an O-haloalkyl group. An example of an haloalkoxy group is OCF₃.

As used herein, the term “haloalkyl” means a C₁₋₆alkyl group having one or more halogen substituents. Examples of haloalkyl groups include, but are not limited to, CF₃, C₂F₅, CH₂F, CHF₂, CCl₃, CHC₁₂, CH₂CF₃, and the like.

As used herein, the term “heteroaryl” means an aromatic heterocycle having up to 20 ring-forming atoms (e.g., C) and having at least one heteroatom ring member (ring-forming atom) such as sulfur, oxygen, or nitrogen. In some embodiments, the heteroaryl group has at least one or more heteroatom ring-forming atoms, each of which are, independently, sulfur, oxygen, or nitrogen. In some embodiments, the heteroaryl group has from 3 to 20 ring-forming atoms, from 3 to 10 ring-forming atoms, from 3 to 6 ring-forming atoms, or from 3 to 5 ring-forming atoms. In some embodiments, the heteroaryl group contains 2 to 14 carbon atoms, from 2 to 7 carbon atoms, or 5 or 6 carbon atoms. In some embodiments, the heteroaryl group has 1 to 4 heteroatoms, 1 to 3 heteroatoms, or 1 or 2 heteroatoms. Heteroaryl groups include monocyclic and polycyclic (e.g., having 2, 3 or 4 fused rings) systems. Examples of heteroaryl groups include, but are not limited to, pyridyl, pyrimidinyl, pyrazinyl, pyridazinyl, triazinyl, furyl, quinolyl, isoquinolyl, thienyl, imidazolyl, thiazolyl, indolyl (such as indol-3-yl), pyrroyl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, pyranyl, oxadiazolyl, isoxazolyl, triazolyl, thianthrenyl, indolizinyl, isoindolyl, isobenzofuranyl, benzoxazolyl, xanthenyl, 2H-pyrrolyl, pyrrolyl, 3H-indolyl, 4H-quinolizinyl, phthalazinyl, naphthyridinyl, quinazolinyl, phenanthridinyl, acridinyl, perimidinyl, phenanthrolinyl, phenazinyl, isothiazolyl, phenothiazinyl, isoxazolyl, furanyl, phenoxazinyl groups, and the like. Suitable heteroaryl groups include 1,2,3-triazole, 1,2,4-triazole, 5-amino-1,2,4-triazole, imidazole, oxazole, isoxazole, 1,2,3-oxadiazole, 1,2,4-oxadiazole, 3-amino-1,2,4-oxadiazole, 1,2,5-oxadiazole, 1,3,4-oxadiazole, pyridine, and 2-aminopyridine.

As used herein, the term “heterocycle” or “heterocyclic ring” means a 5- to 7-membered mono- or bicyclic or 7- to 10-membered bicyclic heterocyclic ring system any ring of which may be saturated or unsaturated, and which consists of carbon atoms and from one to three heteroatoms chosen from N, O and S, and wherein the N and S heteroatoms may optionally be oxidized, and the N heteroatom may optionally be quaternized, and including any bicyclic group in which any of the above-defined heterocyclic rings is fused to a benzene ring. Particularly useful are rings containing one oxygen or sulfur, one to three nitrogen atoms, or one oxygen or sulfur combined with one or two nitrogen atoms. The heterocyclic ring may be attached at any heteroatom or carbon atom which results in the creation of a stable structure. Examples of heterocyclic groups include, but are not limited to, piperidinyl, piperazinyl, 2-oxopiperazinyl, 2-oxopiperidinyl, 2-oxopyrrolodinyl, 2-oxoazepinyl, azepinyl, pyrrolyl, 4-piperidonyl, pyrrolidinyl, pyrazolyl, pyrazolidinyl, imidazolyl, imidazolinyl, imidazolidinyl, pyridyl, pyrazinyl, pyrimidinyl, pyridazinyl, oxazolyl, oxazolidinyl, isoxazolyl, isoxazolidinyl, morpholinyl, thiazolyl, thiazolidinyl, isothiazolyl, quinuclidinyl, isothiazolidinyl, indolyl, quinolinyl, isoquinolinyl, benzimidazolyl, thiadiazoyl, benzopyranyl, benzothiazolyl, benzoxazolyl, furyl, tetrahydrofuryl, tetrahydropyranyl, thienyl, benzothienyl, thiamorpholinyl, thiamorpholinyl sulfoxide, thiamorpholinyl sulfone, and oxadiazolyl. Morpholino is the same as morpholinyl.

As used herein, the term “heterocycloalkyl” means non-aromatic heterocycles having up to 20 ring-forming atoms including cyclized alkyl, alkenyl, and alkynyl groups, where one or more of the ring-forming carbon atoms is replaced by a heteroatom such as an O, N, or S atom. Heterocycloalkyl groups can be mono or polycyclic (e.g., fused, bridged, or spiro systems). In some embodiments, the heterocycloalkyl group has from 1 to 20 carbon atoms, or from 3 to 20 carbon atoms. In some embodiments, the heterocycloalkyl group contains 3 to 14 ring-forming atoms, 3 to 7 ring-forming atoms, or 5 or 6 ring-forming atoms. In some embodiments, the heterocycloalkyl group has 1 to 4 heteroatoms, 1 to 3 heteroatoms, or 1 or 2 heteroatoms. In some embodiments, the heterocycloalkyl group contains 0 to 3 double bonds. In some embodiments, the heterocycloalkyl group contains 0 to 2 triple bonds. Examples of heterocycloalkyl groups include, but are not limited to, morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, oxazolidinyl, isothiazolidinyl, pyrazolidinyl, thiazolidinyl, imidazolidinyl, pyrrolidin-2-one-3-yl, and the like. In addition, ring-forming carbon atoms and heteroatoms of a heterocycloalkyl group can be optionally substituted by oxo or sulfido. For example, a ring-forming S atom can be substituted by 1 or 2 oxo (form a S(O) or S(O)₂). For another example, a ring-forming C atom can be substituted by oxo (form carbonyl). Also included in the definition of heterocycloalkyl are moieties that have one or more aromatic rings fused (having a bond in common with) to the nonaromatic heterocyclic ring including, but not limited to, pyridinyl, thiophenyl, phthalimidyl, naphthalimidyl, and benzo derivatives of heterocycles such as indolene, isoindolene, 4,5,6,7-tetrahydrothieno[2,3-c]pyridine-5-yl, 5,6-dihydrothieno[2,3-c]pyridin-7(4H)-one-5-yl, isoindolin-1-one-3-yl, and 3,4-dihydroisoquinolin-1(2H)-one-3y1 groups. Ring-forming carbon atoms and heteroatoms of the heterocycloalkyl group can be optionally substituted by oxo or sulfido.

As used herein, the term “heterocycloalkylalkyl” means a C1-6 alkyl substituted by heterocycloalkyl.

As used herein, the term “hydroxy” or “hydroxyl” means an OH group.

As used herein, the term “hydroxyalkyl” or “hydroxylalkyl” means an alkyl group substituted by a hydroxyl group. Examples of a hydroxylalkyl include, but are not limited to, —CH₂OH and —CH₂CH₂OH.
the terms “infection” and “bacterial infection” may refer to a gynecological infection. In another aspect the terms “infection” and “bacterial infection” may refer to a respiratory tract infection (RTI). In still another, the terms “infection” and “bacterial infection” may refer to a sexually transmitted disease. In yet another aspect, the terms “infection” and “bacterial infection” may refer to a urinary tract infection (UTI). In a further aspect, the terms “infection” and “bacterial infection” may refer to acute exacerbation of chronic bronchitis (ACEB). In yet a further aspect, the terms “infection” and “bacterial infection” may refer to acute otitis media. In one aspect, the terms “infection” and “bacterial infection” may refer to acute sinusitis. In another aspect, the terms “infection” and “bacterial infection” may refer to an infection caused by drug resistant bacteria. In still another aspect, the terms “infection” and “bacterial infection” may refer to catheter-related sepsis. In yet another aspect, the terms “infection” and “bacterial infection” may refer to chancroid. In a further aspect, the terms “infection” and “bacterial infection” may refer to chlamydia. In still a further aspect, the terms “infection” and “bacterial infection” may refer to community-acquired pneumonia (CAP). In yet a further aspect, the terms “infection” and “bacterial infection” may refer to complicated skin and skin structure infection. In one aspect, the terms “infection” and “bacterial infection” may refer to uncomplicated skin and skin structure infection. In another aspect, the terms “infection” and “bacterial infection” may refer to endocarditis. In still another aspect, the terms “infection” and “bacterial infection” may refer to febrile neutropenia. In yet another aspect, the terms “infection” and “bacterial infection” may refer to gonococcal cervicitis. In a further aspect, the terms “infection” and “bacterial infection” may refer to gonococcal urethritis. In still a further aspect, the terms “infection” and “bacterial infection” may refer to hospital-acquired pneumonia (HAP). In yet another aspect, the terms “infection” and “bacterial infection” may refer to osteomyelitis. In a further aspect, the terms “infection” and “bacterial infection” may refer to sepsis. In still a further aspect, the terms “infection” and “bacterial infection” may refer to syphilis. In a further aspect, the terms “infection” and “bacterial infection” may refer to an intra-abdominal infection (IAI).

In one embodiment of the invention, the terms “infection” and “bacterial infection” refer to a infection caused by Gram-negative bacteria, also referred to as a “Gram-negative infection”. In one aspect of this embodiment, the Gram-negative infection is an infection resistant to one or more antibiotics. In one aspect of this embodiment, the Gram-negative infection is a multi-drug resistant infection.

As used herein, the term “patient,” means any animal, including mammals, such as mice, rats, other rodents, rabbits, dogs, cats, swine, cattle, sheep, horses, or primates, such as humans.

As used herein, the term “isolating” means that separating the compounds described herein from other components of a synthetic organic chemical reaction mixture by conventional techniques, such as filtration.

As used herein, the term “mammal” means a rodent (i.e., a mouse, a rat, or a guinea pig), a monkey, a cat, a dog, a cow, a horse, a pig, or a human. In some embodiments, the mammal is a human.

As used herein, the term “nitro” means —NO₂.

As used herein, the term “n-membered”, where n is an integer, typically describes the number of ring-forming atoms in a moiety, where the number of ring-forming atoms is n. For example, pyridine is an example of a 6-membered heteroaryl ring and thiophene is an example of a 5-membered heteroaryl ring.

As used herein, the phrase “optionally substituted” means that substitution is optional and therefore includes both unsubstituted and substituted atoms and moieties. A “substituted” atom or moiety indicates that any hydrogen on the designated atom or moiety can be replaced with a selection from the indicated substituent groups, provided that the normal valency of the designated atom or moiety is not exceeded, and that the substitution results in a stable compound. For example, if a methyl group is optionally substituted, then 3 hydrogen atoms on the carbon atom can be replaced with substituent groups.

As used herein, the phrase “pharmaceutically acceptable” means those compounds, materials, compositions, and/or dosage forms which are, within the scope of sound medical judgment, suitable for use in contact with tissues of humans and animals. In some embodiments, “pharmaceutically acceptable” means approved by a regulatory agency of the Federal or a state government or listed in the U.S. Pharmacopeia or other generally recognized pharmacopeia for use in animals, and more particularly in humans.

As used herein, the term “zwitterion” refers to a functional group molecule in which at least one has a positive electrical charge and one a negative electrical charge. Provided herein is an invention of the compounds of Formula (I), or a pharmaceutically acceptable salt or zwitterion thereof. The compounds of this invention cover the pharmaceutically acceptable salt or zwitterion forms of the compounds.

In some embodiments, the salt of a compound described herein is a pharmaceutically acceptable salt thereof. As used herein, the phrase “pharmaceutically acceptable salt(s),” includes, but is not limited to, salts of acidic or basic groups. Compounds that are basic in nature are capable of forming a wide variety of salts with various inorganic and organic acids. Acids that may be used to prepare pharmaceutically acceptable acid addition salts of such basic compounds are those that form non-toxic acid addition salts, i.e., salts containing pharmacologically acceptable anions including, but not limited to, sulfuric, thiosulfuric, citric, maleic, acetic, oxalic, hydrochloride, hydrobromide, hydroiodide, nitrate, sulfate, bisulfate, bisulfite, phosphate, acid phosphate, isonicotinate, borate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, bicarbonate, malonate, mesylate, esylate, napsydisylate, tosylate, besylate, orthophoshate, trifluoroacetate, and pamoate (i.e., 1,1′-methylene-bis-(2-hydroxy-3-naphthoate)) salts. Compounds that include an amino moiety may form pharmaceutically acceptable salts with various amino acids, in addition to the acids mentioned above. Compounds that are acidic in nature are capable of forming base salts with various pharmacologically acceptable cations. Examples of such salts include, but are not limited to, alkali metal or alkaline earth metal salts and, particularly, calcium, magnesium, ammonium, sodium, lithium, zinc, potassium, and iron salts. The present embodiments also includes quaternary ammonium salts of the compounds described herein, where the compounds have one or more tertiary amine moiety. Provided herein is an invention of the compounds of Formula (I), or a pharmaceutically acceptable salt or zwitterion thereof. The compounds of this invention cover the pharmaceutically acceptable salt or zwitterion forms of the compounds.

As used herein, the term “phenyl” means —C₆H₅. A phenyl group can be unsubstituted or substituted with one, two, or three suitable substituents.

As used herein, the term “siderophore” is a low molecular weight moiety that can bind ferric iron. Once bound, these “iron carriers” can facilitate transport of the molecule into a bacterial cell. The term “siderophore” is used in accordance with its common meaning and refers to a high-affinity iron chelating compound that may be secreted by a microorganism (e.g., bacteria, fungi, grasses). As defined herein, the siderophore compound may be a synthetic or natural compound.

Synthetic siderophore compounds include synthetic analogs and derivatives of natural siderophore compounds. The term “siderophore moiety” or “Sid” is the partial structure resulting from removal of a terminal —COOH, —NH₂, or —OH group from a free siderophore compound. In some embodiments, the siderophore moiety attaches to the rest part of the compound directed or in the forms of Sid-CO—, Sid-COO—, Sid-NH—, or Sid-O—. In some embodiments, the free siderophore compound, from which the “siderophore moiety” is derived, are represented by Sid-COOH, Sid-NH₂, or Sid-OH.

As used herein, the term “solution” means a liquid composition wherein a first portion of the active agent is present in solution and a second portion of the active agent is present in particulate form, in suspension in a liquid matrix.

As used herein, the term “solvent” means a usually liquid substance capable of dissolving or dispersing one or more other substances including water, inorganic nonaqueous solvent, and organic solvents. The term “inorganic nonaqueous solvent” means a solvent other than water, that is not an organic compound. Examples of the “inorganic nonaqueous solvent” include, but are not limited to: liquid ammonia, liquid sulfur dioxide, sulfuryl chloride and sulfuryl chloride fluoride, phosphoryl chloride, dinitrogen tetroxide, antimony trichloride, bromine pentafluoride, hydrogen fluoride, pure sulfuric acid and other inorganic acids. The term “organic solvent” means carbon-based solvent. Examples of the “organic solvent” include, but are not limited to: aromatic compounds, e.g, benzene and toluene. alcohols, e.g, methanol, ethanol, and propanol, esters and ethers. ketones, e.g, acetone. amines. nitrated and halogenated hydrocarbons. The “organic solvent” includes both polar and non-polar organic solvent. The “polar organic solvent” means an organic solvent that has large dipole moments (aka “partial charges”) and in general the organic solvent with dielectric constants greater than about 5 is considered as “polar organic solvent” while those with dielectric constants less than 5 are considered “non-polar organic solvent.” Examples of the “polar organic solvent” include, but are not limited to, acetic acid, methanol, acetone, and acetonitrile, DMSO, and DMF. Examples of the non-polar organic solvent include, but are not limited to, benzene, carbon tetrachloride, and n-hexane. The “organic solvent” includes both protonic and non-protonic organic solvent. The term “protonic organic solvent” means an organic solvent having a hydrogen atom bonded to oxygen or nitrogen (an acidic hydrogen atom). Examples of the “protonic organic solvent” include, but are not limited to, methanol, ethanol, propanol, isopropanol, butanol, hexanol, phenol, acetic acid, benzoic acid and partly fluorinated compounds thereof. Examples of the “non-protonic organic solvent” include, but are not limited to: ethylene glycol dimethyl ether, ethylene glycol methylethyl ether, diethylene glycol dimethyl ether, diethylene glycol methyl ethyl ether, triethylene glycol dimethyl ether, tetraethylene glycol dimethyl ether, ethylene glycol diethyl ether, diethylene glycol diethyl ether, 1,3-dimethoxypropane, 1,2-dimethoxypropane, propylene glycol dimethyl ether, dipropylene glycol dimethyl ether, dioxane, dimethyl carbonate, ethyl methyl carbonate, diethyl carbonate, ethylene carbonate, propylene carbonate, 2,3-dimethylethylene carbonate, butylene carbonate, acetonitrile, methoxy acetonitrile, propionitrile, butyrolactone, valerolactone, dimethoxyethane, sulforane, methylsulforane, sulfolene, dimethyl sulfone, ethylmethyl sulfone, and isopropyl methyl sulfone.

As used herein, the phrase “suitable substituent” or “substituent” means a group that does not nullify the synthetic or pharmaceutical utility of the compounds described herein or the intermediates useful for preparing them. Examples of suitable substituents include, but are not limited to: C₁-C₆alkyl, C₁-C₆alkenyl, C₁-C₆alkynyl, C₅-C₆aryl, C₁-C₆alkoxy, C₃-C₅heteroaryl, C₃-C₆cycloalkyl, C₅-C₆aryloxy, CN, OH, oxo, halo, haloalkyl, NO₂, CO₂H, NH₂, NH(C₁-C₈alkyl), N(C₁-C₈alkyl)₂, NH(C₆aryl), N(C₅-C₆aryl)₂, CHO, CO(C₁-C₆alkyl), CO((C₅-C₆)aryl), CO₂((C₁-C₆)alkyl), and CO₂((C₅-C₆)aryl). One of skill in art can readily choose a suitable substituent based on the stability and pharmacological and synthetic activity of the compounds described herein.

As used herein, the term “and without limitation” is understood to follow unless explicitly stated otherwise.

At various places in the present specification, substituents of compounds may be disclosed in groups or in ranges. It is specifically intended that embodiments include each and every individual subcombination of the members of such groups and ranges. For example, the term “C₁-C₆ alkyl” is specifically intended to individually disclose methyl, ethyl, propyl, C₄ alkyl, C₅ alkyl, and C₆ alkyl.

For compounds in which a variable appears more than once, each variable can be a different moiety selected from the Markush group defining the variable. For example, where a structure is described having two R groups that are simultaneously present on the same compound, the two R groups can represent different moieties selected from the Markush groups defined for R. In another example, when an optionally multiple substituent is designated in the form, for example, then it is understood that substituent R can occur s number of times on the ring, and R can be a different moiety at each occurrence.

It is further appreciated that certain features described herein, which are, for clarity, described in the context of separate embodiments, can also be provided in combination in a single embodiment. Conversely, various features which are, for brevity, described in the context of a single embodiment, can also be provided separately or in any suitable subcombination.

It is understood that the present embodiments encompass the process, where applicable, of stereoisomers, diastereomers and optical stereoisomers of the compounds, as well as mixtures thereof. Additionally, it is understood that stereoisomers, diastereomers, and optical stereoisomers of the compounds, and mixtures thereof, are within the scope of the embodiments. By way of non-limiting example, the mixture may be a racemate or the mixture may comprise unequal proportions of one particular stereoisomer over the other. Additionally, the compounds can be provided as a substantially pure stereoisomers, diastereomers and optical stereoisomers (such as epimers).

The compounds described herein can be asymmetric (e.g., having one or more stereocenters). All stereoisomers, such as enantiomers and diastereomers, are intended to be included within the scope of the embodiments unless otherwise indicated. Compounds that contain asymmetrically substituted carbon atoms can be isolated in optically active or racemic forms. Methods of preparation of optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are provided herein. Cis and trans geometric isomers of the compounds are also included within the present embodiments and can be isolated as a mixture of isomers or as separated isomeric forms. Where a compound capable of stereoisomerism or geometric isomerism is designated in its structure or name without reference to specific R/S or cis/trans configurations, it is intended that all such isomers are contemplated.

In some embodiments, the composition comprises a compound, or a pharmaceutically acceptable salt thereof, that is at least 90%, at least 95%, at least 98%, or at least 99%, or 100% enantiomeric pure, which means that the ratio of one enantiomer to the other in the composition is at least 90:1 at least 95:1, at least 98:1, or at least 99:1, or is completely in the form of one enantiomer over the other.

Resolution of racemic mixtures of compounds can be carried out by any of numerous methods known in the art, including, for example, chiral HPLC, fractional recrystallization using a chiral resolving acid which is an optically active, salt-forming organic acid. Suitable resolving agents for fractional recrystallization methods include, but are not limited to, optically active acids, such as the D and L forms of tartaric acid, diacetyltartaric acid, dibenzoyltartaric acid, mandelic acid, malic acid, lactic acid, and the various optically active camphorsulfonic acids such as β-camphorsulfonic acid. Other resolving agents suitable for fractional crystallization methods include, but are not limited to, stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like. Resolution of racemic mixtures can also be carried out by elution on a column packed with an optically active resolving agent (e.g., dinitrobenzoylphenylglycine). Suitable elution solvent compositions can be determined by one skilled in the art.

Compounds may also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Examples of prototropic tautomers include, but are not limited to, ketone-enol pairs, amide-imidic acid pairs, lactam-lactim pairs, amide-imidic acid pairs, enamine-imine pairs, and annular forms where a proton can occupy two or more positions of a heterocyclic system including, but not limited to, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds may also include zwitterion forms.

Compounds also include hydrates and solvates, as well as anhydrous and non-solvated forms.

Compounds can also include all isotopes of atoms occurring in the intermediates or final compounds. Isotopes include those atoms having the same atomic number but different mass numbers. For example, isotopes of hydrogen include tritium and deuterium.

In some embodiments, the compounds, or salts thereof, are substantially isolated. Partial separation can include, for example, a composition enriched in the compound. Substantial separation can include compositions containing at least about 50%, at least about 60%, at least about 70%, at least about 80%, at least about 90%, at least about 95%, at least about 97%, or at least about 99% by weight of the compound, or salt thereof. Methods for isolating compounds and their salts are routine in the art.

Although the disclosed compounds are suitable, other functional groups can be incorporated into the compound with an expectation of similar results. In particular, thioamides and thioesters are anticipated to have very similar properties. The distance between aromatic rings can impact the geometrical pattern of the compound and this distance can be altered by incorporating aliphatic chains of varying length, which can be optionally substituted or can comprise an amino acid, a dicarboxylic acid or a diamine. The distance between and the relative orientation of monomers within the compounds can also be altered by replacing the amide bond with a surrogate having additional atoms. Thus, replacing a carbonyl group with a dicarbonyl alters the distance between the monomers and the propensity of dicarbonyl unit to adopt an anti-arrangement of the two carbonyl moiety and alter the periodicity of the compound. Pyromellitic anhydride represents still another alternative to simple amide linkages which can alter the conformation and physical properties of the compound. Modern methods of solid phase organic chemistry (E. Atherton and R. C. Sheppard, Solid Phase Peptide Synthesis A Practical Approach IRL Press Oxford 1989) now allow the synthesis of homodisperse compounds with molecular weights approaching 5,000 Daltons. Other substitution patterns are equally effective.

Embodiments of various processes of preparing compounds of Formula (I) and salts thereof are provided. Where a variable is not specifically recited, the variable can be any option described herein, except as otherwise noted or dictated by context.

In some embodiments, the processes of preparing compounds of Formula (I) or a pharmaceutically acceptable salt thereof is as described in the appended exemplary, non-limiting claims.

Provided herein is an invention directed to a compound of Formula (I):

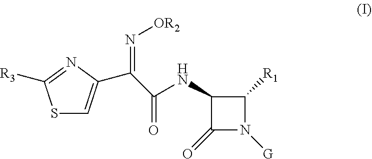

or a pharmaceutically acceptable salt or zwitterion thereof, wherein
G is —OR^{A}, —OCH₂C(═O)X, OCHRC(═O)X, —C(═O)X, —S(═O)₂X, —OS(═O)₂X, —C(═O)NH—S(═O)₂G′, P(═O)X or —P(═O)X,
G′ is OR^{A}, —OCH₂C(═O)X, OCHRC(═O)X, —C(═O)X, —S(═O)₂X, —OS(═O)₂X, P(═O)X or —P(═O)X,
X is H, —OR^{A} or —N(R^{A})₂,
R₁ is H, or —(C₁-C₁₂)alkyl optionally substituted by X or Z,
Z is R^{A}, OR^{A}, —SR^{A}, —C(═O)N(R^{A})₂, or —N(R^{A})₂,
R₂ is —CH₂C(═O)W, —CHR⁴C(═O)W, —C(R⁴)₂C(═O)W, —C(R⁴)₂C(═O)NHOCH₂C(═O)W, —C(R⁴)₂C(═O)NHOCH₂C(═O)OH, —C(R⁴)₂C(═O)NHOC(R⁴)₂C(═O)OH, or —C(R⁴)₂C(═O)NHOC(R⁴)₂C(═O)W,
R₃ is H, Z, or R⁴,
each R⁴ is independently H, Z, —(C₁-C₁₂)alkyl, —(C₃-C₈)cycloalkyl, —(C₃-C₆)heterocycloalkyl, or —(C₁-C₈)N(R^{A})R^{B}, or two R⁴ are taken together with the carbon atom to which both are attached to form a —(C₃-C₈)cycloalkyl or —(C₃-C₆)heterocycloalkyl,
each R^{A} is independently H, —(C₁-C₁₂)alkyl, —(C₃-C₈)cycloalkyl, phenyl, aryl, or heteroaryl,
R^{B} is H, —(C₁-C₁₂)alkyl, —(C₃-C₈)cycloalkyl, or phenyl,
W is Sid or linker-Sid,
Sid is a siderophore moiety,
linker is —NH-Q-C(═O)O—, —NH—C(═O)-Q-C(═O)O—, —C(═O)-Q-, —C(═O)O-Q-, —C(═O)-Q-C(═O)—NH—, —O-Q-O—, —O-Q-, —NH-Q-, —NH-Q-NH—, —NH-Q-O—, —NH—O-Q-, or the combination thereof,
Each Q is independently a —(C₁-C₁₂)alkylene optionally substituted by one or more selected from the group consisting of —OH, —COOH, ═O, —NH₂, —(C₁-C₁₂)alkenyl, —(C₁-C₁₂)alkylene —OH, —(C₁-C₁₂)alkylene —NH₂, —NH—(C₁-C₁₂)alkylene-COOH, —NH—C(═O)—(C₁-C₁₂)alkylene —COOH, —C(═O)—(C₁-C₁₂)alkylene —C(═O)—NH₂, —O—(C₁-C₁₂)alkylene-OH, —NH—(C₁-C₁₂)alkylene —NH₂, and —NH—(C₁-C₁₂)alkylene —OH.

In some embodiments, Sid-OH, Sid-NH₂, Sid-COOH, or -linker-Sid is represented by Formula (II):

R₈—U₁-R₉ (II)

wherein
U₁ is a covalent bond, or optionally substituted -Q-, —C(═O)—, —O—C(═O)—, —CHR₈—, —CR₁₀R₈—, —C(═O)—NH—, —C(═O)—NR₈—, —C(═O)—N(OH)—, —NH—, —NR₈—, —NR₁₀—, —NH—CHR₈—C(═O)—, —R₁₁—, or the combination thereof,
each R₈ is independently H, —OH, —COOH, —NH₂, -Q-OH, —V, -Q-V, —NH—C(═O)—V, -Q-NH—C(═O)—V, —NR₁₀—C(═O)—V, —NR₁₀—V, —C(═O)—V, —O—C(═O)—V, —C(═O)—R₁₁—V, —NH—C(═O)—R₁₁—V, —C(═O)—R₁₁-aryl, or linker-H,
R₉ is H, —OH, —COOH, —NH₂, -Q-OH, —V, -Q-V, —NH—C(═O)—V, -Q-NH—C(═O)—V, —NR₁₀—C(═O)—V, —NR₁₀—V, —C(═O)—V, —O—C(═O)—V, —C(═O)—R₁₁—V, —NH—C(═O)—R₁₁—V, —C(═O)—R₁₁-aryl, or linker-H,
or R₈ and R₉ are taken together as a covalent bond to form a ring,
R₁₀ is —NH—C(O)-Q-COOH, —C(O)-Q-COOH, -Q-COOH, or —COOH,
R₁₁ is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S,
V═—N(OH)—C(═O)—NH₂, —N(OH)—C(═O)H, —N(OH)—C(═O)— linker, —N(OH)—C(═O)—(C₁-C₁₂)alkyl, —N(OH)—C(═O)—(C₁-C₁₂)alkenyl, —N(OH)—C(═O)-Q-OH, —N(OH)—C(═O)-Q-COOH,

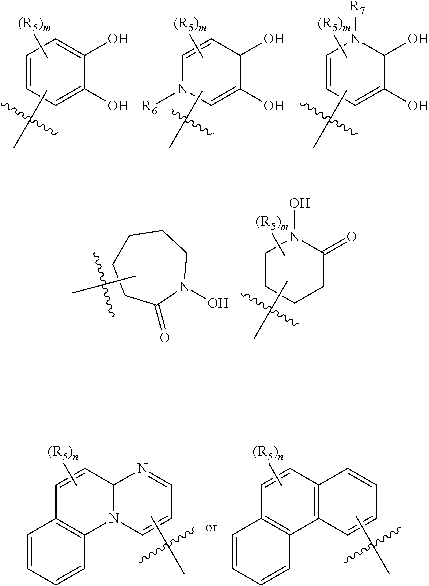

m is an integral selected from 0-3,
n is an integral selected from 0-10,
each R₅ is independently —(C₁-C₁₂)alkyl, halogen, —OH, —COOH, —NH, -linker-H, or —C(═O)— linker-H, and
R₆ and R₇ are independently H, —COOH, or —OR^{A}.

In some embodiments, a compound of Formula (I) is represented by

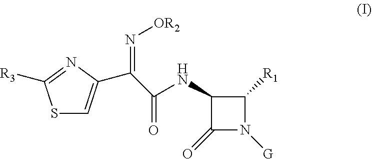

or a salt or zwitterion thereof;
wherein
G is OR^{A}, —OCH₂C(═O)X, OCHRC(═O)X, —C(═O)X, —S(═O)₂X, —OS(═O)₂X, —P(═O)X₂ or —P(═O)X₂, wherein X is —OR^{A} or —N(R^{A})₂,
R¹ is H, or —(C₁-C₁₂)alkyl optionally substituted by X or Z,
Z is —OR^{A}, SR^{A}, or —N(R^{A})₂,
R₂ is CH₂C(═O)W, CHR⁴C(═O)W, C(R⁴)₂C(═O)W; C(R⁴)₂C(═O)NHOCH₂C(═O)W, C(R⁴)₂C(═O)NHO C(R⁴)₂C(═O)W,
R⁴ is Z or H, —(C₁-C₁₂)alkyl, —(C₃-C₈)cycloalkyl, —(C₃-C₆)heterocycloalkyl, or —(C₁-C₈)N(R^{A})R^{B},
each R^{A} is independently H, —(C₁-C₁₂)alkyl, —(C₃-C₈)cycloalkyl, phenyl, aryl or heteroaryl;
R^{B} is H, —(C₁-C₁₂)alkyl, —(C₃-C₈)cycloalkyl, phenyl,
W is a siderophore (Sid) or linker-siderophore,
R₃ is H, Z or R⁴, and
linker is NH—R¹—NH, O—R¹—NH, or O—R¹—O.

In some embodiments, the compound of Formula (I) has a part of the structure that is moiety of oxamazins (7), monosulfactams (8), monobactams (9) and monocarbams (10).

In some embodiments, U₁ is

—U₁₁—U₁₂—U₁₃—U₁₄—U₁₅—U₁₆— (II-1)

wherein
U₁₁, U₁₂, U₁₃, U₁₄, U₁₅, and U₁₆ are independently a covalent bond, or optionally substituted -Q-, —C(═O)—, —O—C(═O)—, —CHR₈—, —CR₁₀R₈—, —C(═O)—NH—, —C(═O)—NR₈—, —C(═O)—N(OH)—, —NH—, —NR₈—, —NR₁₀—, —NH—CHR₈—C(═O)—, or —R₁₁—.

In some embodiments, G is O—CH₂—COOH, S(═O)₂OH, OS(═O)₂OH, or C(═O)NH—S(═O)₂G′.

In some embodiments, G is S(═O)₂OH.

In some embodiments, R₁ is —(C₁-C₁₂)alkyl.

In some embodiments, R₁ is —CH₃.

In some embodiments, R₂ is —C(R⁴)₂C(═O)W, C(R⁴)₂C(═O)NHOCH₂C(═O)OH, or —C(R⁴)₂C(═O)NHOC(R⁴)₂C(═O)W.

In some embodiments, R⁴ is —CH₃.

In some embodiments, R₃ is —NH₂.

In some embodiments, Formula (I) is represented by

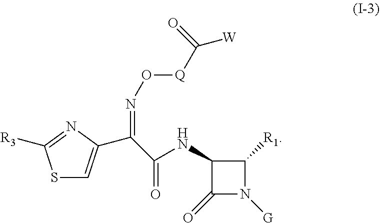

In some embodiments, Formula (I) is represented by

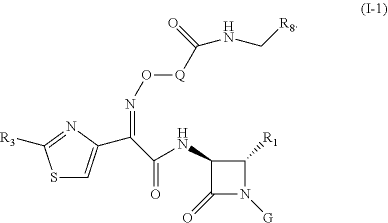

In some embodiments, Formula (I) is represented by

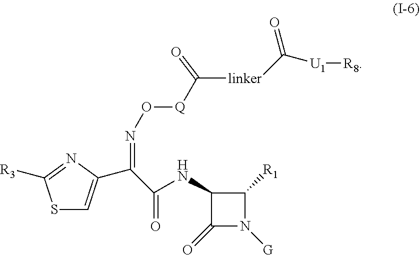

In some embodiments, Formula (I) is represented by

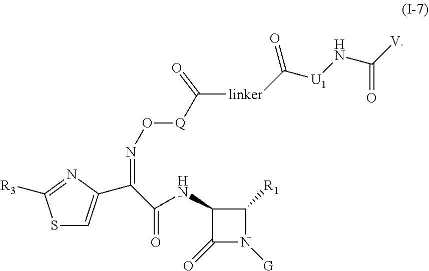

In some embodiments, Formula (I) is represented by

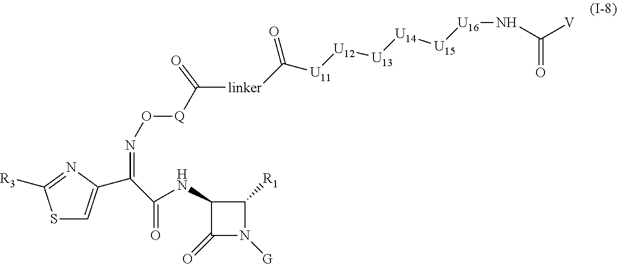

In some embodiments, Formula (I) is represented by

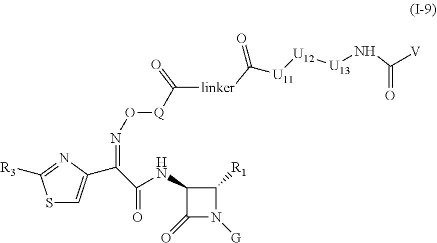

In some embodiments, Formula (I) is represented by

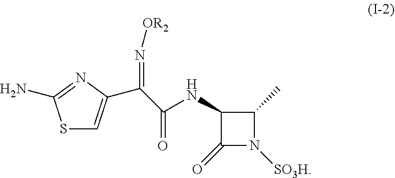

In some embodiments, V is

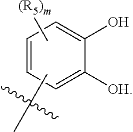

In some embodiments, linker is —NH-Q-NH— or —NH—O-Q, or the combination thereof. In some embodiments, linker is —NH—CH₂CH₂—NH— or —NH—O—CH₂CH₂—, —NH—O—CH₂C(═O)—, or the combination thereof.

In some embodiments, R₈ or R₉ is COOH. In some embodiments, one of R₈ and R₉ is COOH, and the other one of R₈ and R₉ is —NH—C(═O)—V.

In some embodiments, W is represented by —C(═O)—U₁-R₈ or —NH-U₁-R₈. In some embodiments, W is represented by -linker-U₁-R₈. In some embodiments, W is represented by -linker-C(═O)—U₁-R₈, In some embodiments, W is represented by —NH-U₁-R₈.

In some embodiments, U₁ is -Q-NR₈-Q- and each Q is independently a —(C₁-C₁₂)alkylene optionally substituted by one or more selected from the group consisting of —OH, —COOH, ═O, and —NH₂.

In some embodiments, W is Sid and Sid-COOH or Sid-OH is represented by R₈—U₁-R₉, wherein
R₈ is —NH—C(═O)—V,
R₉ is —COOH,
U₁ is -Q-NR₈-Q-
V is

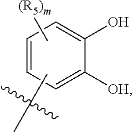

R₅ is Cl, and
m is 0 or 1.

In some embodiments, W is represented by —C(═O)—U₁-R₈ or —NH—U₁-R₈, wherein
R₈ is —NH—C(═O)—V,
U₁ is -Q-NR₈-Q-
V is

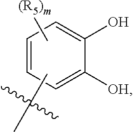

R₅ is Cl, and
m is 0 or 1.

In some embodiments, Sid is derived from catecholates, hydroxamates, carboxylates, ferrichrome, deferoxamine, desferrioxamine, fusarinine C, ornibactin, rhodotorulic acid, enterobactin, bacillibactin, vibriobactin, azotobactin, pyoverdine, yersiniabactin, aerobactin, simochelin, alcaligin, mycobactin, staphyloferrin A, or petrobactin. In some embodiments, Sid is derived from Achromobactin, Acinetobactin, Acinetoferrin, Aerobactin, Aeruginic, Agrobactin, Agrobactin A, Albomycin 271, Alcaligin 230, Alterobactin A, Alterobactin B, Aminochelin 262, Amonabactin P693, Amonabactin P750, Amonabactin T732, Amonabactin T789, Amphibactin B, Amphibactin C, Amphibactin D, Amphibactin E, Amphibactin F, Amphibactin G, Amphibactin H, Amphibactin I, Amycolachrome 235, Anachelin 1, Anachelin 2, Anguibactin 247, Aquachelin A, Aquachelin B, Aquachelin C, 2, Aquachelin D, Arthrobactin, Arthrobactin 199, Asperchrome A, Asperchrome B1, Asperchrome B2, Asperchrome B3, Asperchrome C, Asperchrome D1, Asperchrome D2, Asperchrome D3, Asperchrome E, Asperchrome F1, Asperchrome F2, Asperchrome F3, Aspergillic acid, Avenic acid, Awaitin A, Awaitin B, Awaitin C, Azotobactin 236, Azotobactin D, Azotobactin 87, Azotochelin, Azotochelin 236, Azoverdin 174, Bacillibactin 85, Basidiochrome 46, Biscatechol, Bisucaberin 232, Carboxymycobactin 107, Carboxymycobactin 1, Carboxymycobactin 2, Carboxymycobactin 3, Carboxymycobactin 4, Cepabactin 266, Chrysobactin 261, Citrate 260, Coelichelin 72, 3, Coprogen 51, Coprogen B, Corynebactin 84, Danoxamine, Deoxydistichonic acid, 2′-Deoxymugineic acid, Deoxyschizokinen 251, Des(diserylglycyl)-ferrirhodin 45, Desacetylcoprogen 52, Desferrioxamine A1, Desferrioxamine A2, Desferrioxamine B, Desferrioxamine D1, Desferrioxamine D2, Desferrioxamine E, Desferrioxamine Etl 21A, Desferrioxamine Et2 21B, Desferrioxamine Et3 21C, Desferrioxamine G1, Desferrioxamine G2A, Desferrioxamine G2B, Desferrioxamine G2C, Desferrioxamine H, Desferrioxamine P1, Desferrioxamine T1, Desferrioxamine T2, Desferrioxamine T3, Desferrioxamine T7, Desferrioxamine T8, Desferrioxamine Tel 21D, Desferrioxamine Te2 21E Desferrioxamine Te3 21F, Desferrioxamine X1, Desferrioxamine X2, 4, Desferrioxamine X3, Desferrioxamine X4, Desferrithiocin, Diamine biscatechol, Dihydrobenzolate, 2,3-Dihydroxybenzoylserine, Dimerum acid, Dimethylcoprogen, Dimethylneocoprogen I, Dimethyltriornicin, Distichonic acid, Enantio Rhizoferrin, Enantio-Pyochelin, Enterobactin, Enterochelin, Exochelin MN, Exochelin MS, Ferrichrome, Ferrichrome A, Ferrichrome C, Ferrichrysin, Ferricrocin, Ferrioxamine, Ferrimycin A, Ferrirhodin, Ferrirubin, Ferrocin A, Fimsbactin A, Fluvibactin, Formobactin, Foroxymithine, Fusarinine A, Fusarinine B, Fusarinine C, Heterobactin A, Heterobactin B, Hydroxycopropen, Hydroxypyridone, Hydroxyisoneocoprogen I, 3-Hydroxymugineic acid, 5, Hydroxy-neocoprogen I, Isoneocoprogen I, Isopyoverdin BTP1, Isopyoverdin 6.7, Isopyoverdin 7.13, Isopyoverdin 90-33, Isopyoverdin 90-44, Isopyoverdin 10.7, Isotriornicin, Itoic acid, Loihichelin A, Loihichelin B, Loihichelin C, Loihichelin D, Loihichelin E, Loihichelin F, Maduraferrin, Malonichrome, Marinobactin A, Marinobactin B, Marinobactin C, Marinobactin D1, Marinobactin D2, Marinobactin E, Micacocidin, Mugineic acid, Mycobactin, Mycobactin A, Mycobactin Av, Mycobactin F, Mycobactin H, Mycobactin J, Mycobactin M, Mycobactin N, 6, Mycobactin NA, Mycobactin P, Mycobactin R, Mycobactin S, Mycobactin T, Myxochelin, ω-N-acetyl-ω-N-hydroxyl-α-aminoalkane, ε-N-acetyl-ε-N-hydroxyl L-lysine, δ-N-acetyl-δ-N-hydroxyl L-ornithine, Nannochelin A, Nannochelin B, Nannochelin C, Neocoprogen I, Neocoprogen II, Neurosporin, Nocobactin, Nocobactin NA, Ochrobactin A, Ochrobactin B, Ochrobactin C, Ornibactin-C4, Ornibactin-C6, Ornibactin-C8, Ornicorrugatin, palmitoylcoprogen, Parabactin, Parabactin A, Petrobactin, Petrobactin disulphonate, Petrobactin sulphonate, Pistillarin, Polyamine biscatechol, Protochelin, Pseudoalterobactin A, Pseudoalterobactin B, Pseudobactin 112, Pseudobactin 589A, 7, Putrebactin, Pyochelin, Pyoverdin A214, Pyoverdin BTP2, Pyoverdin C, Pyoverdin CHAO, Pyoverdin D-TR133, Pyoverdin E, Pyoverdin G R Pyoverdin GM, Pyoverdin I-III, Pyoverdin P19, Pyoverdin Pau, Pyoverdin PL8, Pyoverdin PVD, Pyoverdin R′, Pyoverdin Thai, Pyoverdin TII, Pyoverdin 1, Pyoverdin 11370, Pyoverdin 13525, Pyoverdin 1547, Pyoverdin 17400, Pyoverdin 18-1, Pyoverdin 19310, Pyoverdin 2192, Pyoverdin 2392, Pyoverdin 2461, Pyoverdin 2798, Pyoverdin 51W, Pyoverdin 9AW, Pyoverdin 90-51, Pyoverdin 95-275, Pyoverdin 96-312, Pyoverdin 96-318, Pyoverdin, Pyoverdin 6.1, Pyoverdin 6.2, Pyoverdin 6.3, Pyoverdin 6.4, Pyoverdin 6.5, Pyoverdin 6.6, Pyoverdin 6.8, Pyoverdin 7.1, Pyoverdin 7.2, Pyoverdin 7.3, Pyoverdin 7.4, Pyoverdin 7.5, Pyoverdin 7.6, Pyoverdin 7.7, Pyoverdin 7.8, Pyoverdin 7.9, Pyoverdin 7.10, Pyoverdin 7.11, Pyoverdin 7.12, Pyoverdin 7.14, Pyoverdin 7.15, Pyoverdin 7.16, Pyoverdin 7.17, Pyoverdin 7.18, Pyoverdin 7.19, Pyoverdin 8.1, Pyoverdin 8.2, Pyoverdin 8.3, Pyoverdin 8.4, Pyoverdin 8.5, Pyoverdin 8.6, Pyoverdin 8.7, Pyoverdin 8.8, Pyoverdin 8.9, Pyoverdin 9.1, Pyoverdin 9.2, Pyoverdin 9.3, Pyoverdin 9.4, Pyoverdin 9.5, Pyoverdin 9.6, Pyoverdin 9.7, Pyoverdin 9.8, Pyoverdin 9.9, Pyoverdin 9.10, Pyoverdin 9.11, Pyoverdin 9.12, Pyoverdin 10.1, Pyoverdin 10.2, Pyoverdin 10.3, Pyoverdin 10.4, Pyoverdin 10.5, Pyoverdin 10.6, Pyoverdin 10.8, Pyoverdin 10.9, Pyoverdin 10.10, Pyoverdin 11.1, Pyoverdin 11.2, Pyoverdin 12, Pyoverdin 12.1, Pyoverdin 12.2, Pyoverdine, Pyridoxatin, Quinolobactin, Rhizobactin, 10, Rhizobactin, Rhizoferrin, Rhizoferrin analogues 88A-88E, Rhodotrulic acid, Salmochelin S1, Salmochelin S2, Salmochelin S4, Salmochelin SX, Salmycin A, Schizokinen, Serratiochelin, Siderochelin A, Snychobactin A, Snychobactin B, Snychobactin C, Staphyloferrin A, Staphyloferrin B, Tetraglycine ferrichrome, Thiazostatin, Triacetylfusarinine, Tricatechol, Triornicin, Vibriobactin, Vibrioferrin, Vicibactin, Vulnibactin, or Yersiniabactin. In some embodiments, Sid is derived from Aerobactin, Agrobactin, Arthrobactin, Awaitin A, Awaitin B, Awaitin C, Azotochelin, Biscatechol, Danoxamine, Dihydrobenzolate, Enterobactin, Ferricrocin, Ferrioxamine, Fimsbactin A, Foroxymithine, Hydroxypyridone, Mycobactin, ω-N-acetyl-ω-N-hydroxyl-α-aminoalkane, ε-N-acetyl-ε-N-hydroxyl L-lysine, δ-N-acetyl-δ-N-hydroxyl L-ornithine, Parabactin, Pyoverdine, Rhodotrulic acid, Schizokinen, or Tricatechol.

Also provided herein is an invention directed to a pharmaceutical composition comprising the compound of Formula (I) or a pharmaceutically acceptable salt or zwitterion thereof, and at least one pharmaceutically acceptable carrier, diluent, or excipient.

In some embodiments, the compound of Formula (I) is selected from the group consisting of:

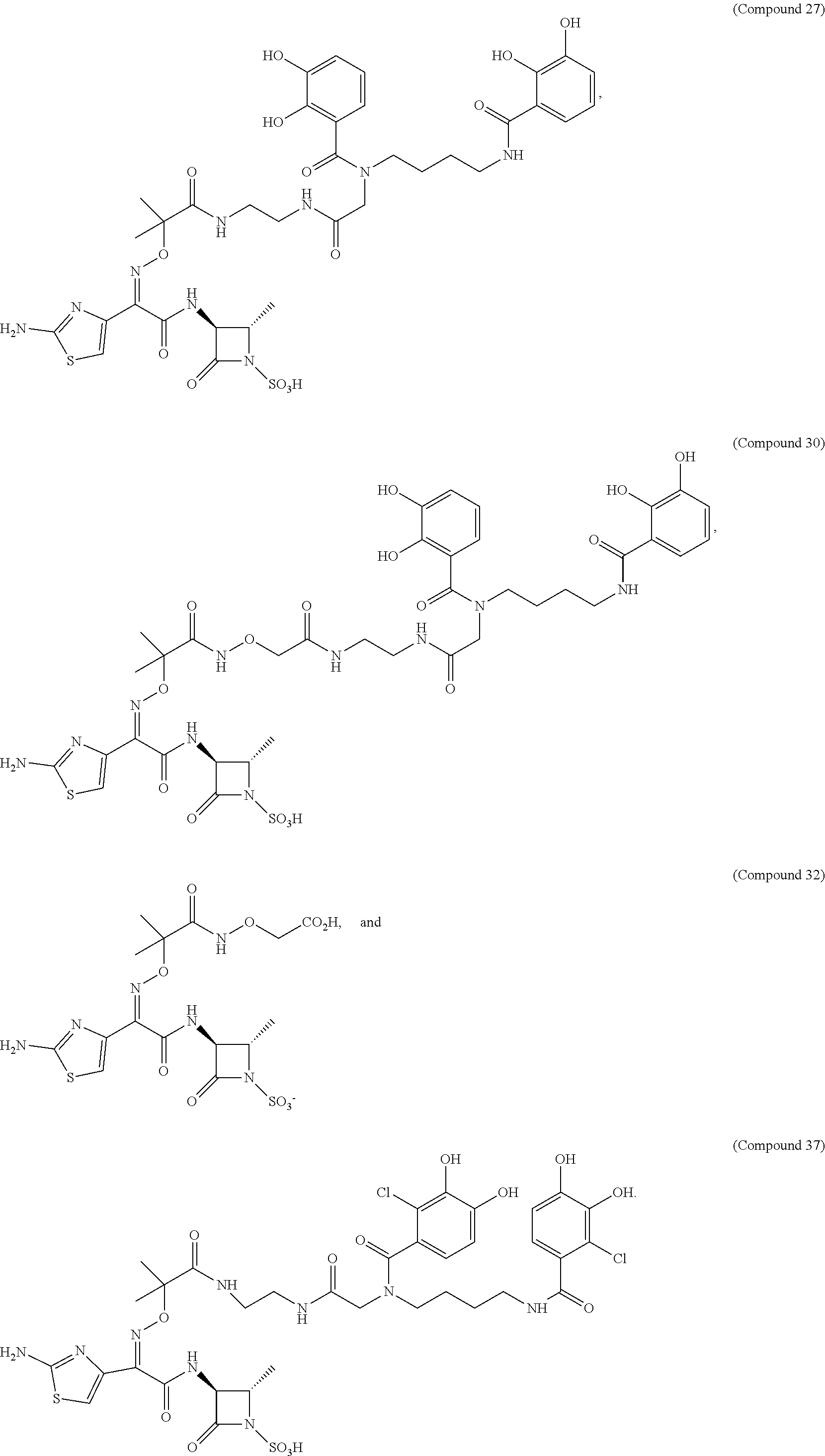

Further provided herein is an invention of a method of treating a bacterial infection in a subject in need thereof, comprising administering to the said subject a therapeutically effective amount of the compound of Formula (I) or a pharmaceutically acceptable salt or zwitterion thereof or a therapeutically effective amount of the pharmaceutical composition comprising therapeutically effective amount of the compound of Formula (I).

In some embodiments, the bacterial infection is a gram-negative bacterial infection. In some embodiments, the bacterial infection is caused by Pseudomonas aeruginosa, Acinetobacter baumannii, Escherichia coli and Klebsiella pneumonia.

In some embodiments, the subject is a human subject. In some embodiments, the subject is an animal subject. In some embodiments, the subject is a mammal subject.

In some embodiments, the method of treating a bacterial infection further comprises administering an effective amount of an additional antibiotic agent. In some embodiments, the additional antibiotic compound is selected from the group consisting of penicillin, methicillin, oxacillin, nafcillin, cloxacillin, dicloxacillin, flucloxacillin, temocillin, amoxicillin, ampicillin, co-amoxiclav, azlocillin, carbenicillin, ticarcillin, mezlocillin, piperacillin, cephalexin, cephalothin, CXA-101, cefazolin, cefaclor, cefuroxime, cefamandole, cefotetan, cefoxitin, ceftriaxone, cefotaxime, cefpodoxime, cefixime, ceftazidime, ceftobiprole medocaril, cefepime, cefpirome, ceftaroline, imipenem, meropenem, ertapenem, faropenem, sulopenem, doripenem, PZ-601 (Protez Pharmaceuticals), ME1036 (Forest Labs), BAL30072, MC-1, tomopenem, tebipenemn, aztreonam, tigemonam, nocardicin A, or tabtoxinine-β-lactam.

In some embodiments, the bacterial infection is resistant to one or more antibiotics.

In some embodiments, the bacterial infection causes a disease selected from the group consisting of urinary tract infections, pneumonia, prostatitis, skin and soft tissue infections, sepsis, and intra-abdominal infections.

Further provided herein is an invention directed to a process of preparing a compound of Formula (I), or a pharmaceutically acceptable salt or zwitterion thereof, the process comprising: contacting a compound of Formula (I′-1)

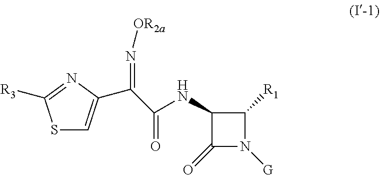

with a compound of Formula (II′-1) or Formula (II′-2)

R₈—U₁—NH₂, (II′-1), or

R₈—U₁-L′—NH₂ (II′-2)

under suitable conditions to produce the compound having Formula (I)

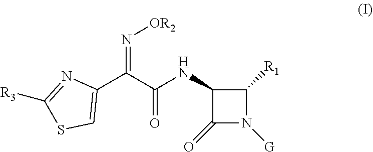

wherein
G is —OR^{A}, —OCH₂C(═O)X, OCHRC(═O)X, —C(═O)X, —S(═O)₂X, —OS(═O)₂X, —C(═O)NH—S(═O)₂G′, P(═O)X or —P(═O)X,
G′ is OR^{A}, —OCH₂C(═O)X, OCHRC(═O)X, —C(═O)X, —S(═O)₂X, —OS(═O)₂X, P(═O)X or —P(═O)X,
X is H, —OR^{A} or —N(R^{A})₂,
R₁ is H, or —(C₁-C₁₂)alkyl optionally substituted by X or Z,
Z is R^{A}, OR^{A}, —SR^{A}, —C(═O)N(R^{A})₂, or —N(R^{A})₂,
R₂ₐ is —CH₂C(═O)OH, —CHR⁴C(═O)OH, —C(R⁴)₂C(═O)OH, or —C(R⁴)₂C(═O)NHOCH₂C(═O)OH, —C(R⁴)₂C(═O)NHOC(R⁴)₂C(═O)OH,
R₂ is —CH₂C(═O)W′, —CHR⁴C(═O)W′, —C(R⁴)₂C(═O)W′, —C(R⁴)₂C(═O)NHOCH₂C(═O)W′, —C(R⁴)₂C(═O)NHOCH₂C(═O)OH, —C(R⁴)₂C(═O)NHOC(R⁴)₂C(═O)OH, or —C(R⁴)₂C(═O)NHOC(R⁴)₂C(═O)W′,
R₃ is H, Z, or R⁴,
each R⁴ is independently H, Z, —(C₁-C₁₂)alkyl, —(C₃-C₅)cycloalkyl, —(C₃-C₆)heterocycloalkyl, or —(C₁-C₈)N(R^{A})R^{B}, or two R⁴ are taken together with the carbon atom to which both are attached to form a —(C₃-C₅)cycloalkyl or —(C₃-C₆)heterocycloalkyl,
each R^{A} is independently H, —(C₁-C₁₂)alkyl, —(C₃-C₅)cycloalkyl, phenyl, aryl, or heteroaryl,
R^{B} is H, —(C₁-C₁₂)alkyl, —(C₃-C₅)cycloalkyl, or phenyl,
each R⁴ is independently H, Z, —(C₁-C₁₂)alkyl, —(C₃-C₅)cycloalkyl, —(C₃-C₆)heterocycloalkyl, or —(C₁-C₈)N(R^{A})R^{B},
each R^{A} is independently H, —(C₁-C₁₂)alkyl, —(C₃-C₈)cycloalkyl, phenyl, aryl, or heteroaryl,
R^{B} is H, —(C₁-C₁₂)alkyl, —(C₃-C₈)cycloalkyl, or phenyl, W′ is —NH-U₁-R₈ or —NH-L′—U₁-R₈,
L′ is -Q-C(═O)O—, —C(═O)-Q-C(═O)O—, —C(═O)-Q-, —C(═O)O-Q-, —C(═O)-Q-C(═O)—, —O-Q-, -Q-, -Q-NH—, -Q-O—, or the combination thereof,
each Q is independently a —(C₁-C₁₂)alkylene optionally substituted by one or more selected from the group consisting of —OH, —COOH, ═O, —NH₂, —(C₁-C₁₂)alkyl, —(C₁-C₁₂)alkenyl, —(C₁-C₁₂)alkylene —OH, —(C₁-C₁₂)alkylene —NH₂, —NH—(C₁-C₁₂)alkylene-COOH, —NH—C(═O)—(C₁-C₁₂)alkylene —COOH, —C(═O)—(C₁-C₁₂)alkylene —C(═O)—NH₂, —O—(C₁-C₁₂)alkylene-OH, —NH—(C₁-C₁₂)alkylene —NH₂, and —NH—(C₁-C₁₂)alkylene —OH.
U₁ is a covalent bond, or optionally substituted -Q-, —C(═O)—, —O—C(═O)—, —CHR₈—, —
CR₁₀R₈—, —C(═O)—NH—, —C(═O)—NR₈—, —C(═O)—N(OH)—, —NH—, —NR₈—, —NR₁₀—, —NH—CHR₈—
C(═O)—, —R₁₁—, or the combination thereof,
each R₈ is each independently H, —OH, —COOH, —NH₂, -Q-OH, —V, -Q-V, —NH—C(═O)—V, -Q-NH—C(═O)—V, —NR₁₀—C(═O)—V, —NR₁₀—V, —C(═O)—V, —O—C(═O)—V, —C(═O)—R₁₁—V, —NH—C(═O)—R₁₁—V, —C(═O)—R₁₁-aryl, or L′—H,
R₁₀ is —NH—C(O)-Q-COOH, —C(O)-Q-COOH, -Q-COOH, or —COOH,
R₁₁ is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S,
V═—N(OH)—C(═O)—NH₂, —N(OH)—C(═O)H, N(OH)—C(═O)-L′, —N(OH)—C(═O)—(C₁-C₁₂)alkyl, —N(OH)—C(═O)—(C₁-C₁₂)alkenyl, —N(OH)—C(═O)-Q-OH, —N(OH)—C(═O)-Q-COOH,

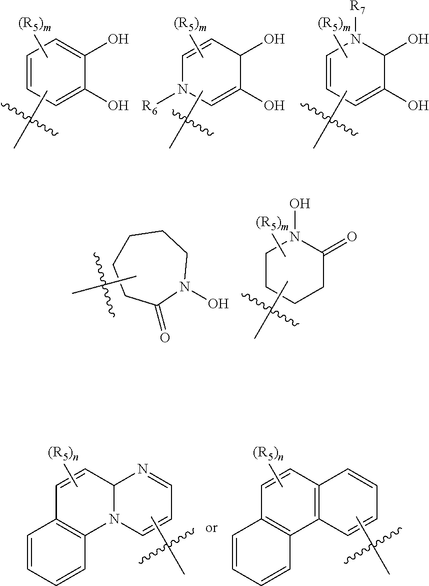

m is an integral selected from 0-3,
n is an integral selected from 0-10,
each R₅ is independently —(C₁-C₁₂)alkyl, halogen, —OH, —COOH, —NH₂, -L′-H, or —C═)-L′-H, and
R₆ and R₇ are independently H, —COOH, or —OR^{A}.

In some embodiments, the process of preparing a compound of Formula (I) comprises coupling the compound of (I′-1) with the compound of Formula (II′-1) or Formula (II′-2) to produce the compound of Formula (I).

In some embodiments, the process of preparing a compound of Formula (I) comprises coupling at room temperature.

In some embodiments, the process of preparing a compound of Formula (I) comprises coupling which comprises contacting the compound of (I′-1) and the compound of Formula (II′-1) or Formula (II′-2) with a coupling reagent.

In some embodiments, the process of preparing a compound of Formula (I) comprises the steps of
(a) Mixing the compound of Formula (I′-1) in a solvent with a coupling reagent,
(b) stirring to obtain a solution,
(c) Mixing the compound of Formula (II′-1) or (II′-2) in a solvent with the solution of step (b), optionally adding additional coupling reagent,
(d) stirring, and
(e) removing the solvent to obtain the compound of Formula (I).

In some embodiments of the above process of preparing a compound of Formula (I), the stirring at step (b) or step (d) is at room temperature. In some embodiments, step (e) comprises reduced pressure evaporation. In some embodiments, the solvent in step (a) or step (c) is water, tetrahydrofuran (THF), dimethylformamide (DMF), or a mixture thereof.

In some embodiments, the process of preparing a compound of Formula (I), comprises the steps of
(a) Mixing the compound of Formula (I′-1) and the compound of Formula (II′-1) or Formula (II′-2) in a solvent to from a solution,
(b) Maintaining or adjusting the pH of the solution,
(c) Dissolving a coupling reagent in a solvent, and
(d) Mixing the solution of step (c) and the solution of step (b) while maintaining or adjusting the pH of the solution, and
(e) obtaining the compound of Formula (I).

In some embodiments of the above process of preparing a compound of Formula (I), the pH of the solution in step (b) or step (d) is about 4.5. In some embodiments, the coupling reagent is HBTU, DIPEA, N-hydroxysuccinimide, EDC-HCl, or mixture thereof. In some embodiments.

In some embodiments, the process of preparing a compound of Formula (I) comprises contacting a compound of Formula (I′-1), wherein in the compound of Formula (I′-1), G is S(═O)₂OH.

In some embodiments, the process of preparing a compound of Formula (I) comprises contacting a compound of Formula (I′-1), wherein in the compound of Formula (I′-1), R₁ is —(C₁-C₁₂)alkyl. In some embodiments, R₁ is —CH₃.

In some embodiments, the process of preparing a compound of Formula (I) comprises contacting a compound of Formula (I′-1) with a compound of Formula (II′-1) or Formula (II′-2), wherein in the compound of Formula (II′-1) or Formula (II′-2), V is

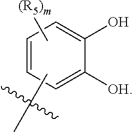

In some embodiments, the process of preparing a compound of Formula (I) comprises contacting a compound of Formula (I′-1) with a compound of Formula (II′-1) or Formula (II′-2), wherein in the compound of Formula (II′-1) or Formula (II′-2), L′—NH₂ is —NH-Q-NH₂ or -Q-O—NH₂, or the combination thereof. In some embodiments, L′—NH₂ is —NH—CH₂CH₂—NH₂, —CH₂CH₂—ONH₂, —C(═O)CH₂—ONH₂, or the combination thereof.

In some embodiments, the process of preparing a compound of Formula (I) comprises contacting a compound of Formula (I′-1) with a compound of Formula (II′-1) or Formula (II′-2), wherein in the compound of Formula (II′-1) or Formula (II′-2), R₈ is —NH—C(═O)—V.

In some embodiments, the process of preparing a compound of Formula (I) comprises contacting a compound of Formula (I′-1) with a compound of Formula (II′-1) or Formula (II′-2), wherein in the compound of Formula (II′-1) or Formula (II′-2), U₁ is -Q-NR₈-Q- and each Q is independently a —(C₁-C₁₂)alkylene optionally substituted by one or more selected from the group consisting of —OH, —COOH, ═O, and —NH₂.

In some embodiments, the process of preparing a compound of Formula (I) comprises contacting a compound of Formula (I′-1) with a compound of Formula (II′-1) or Formula (II′-2), wherein in the compound of Formula (II′-1) or Formula (II′-2),
R₈ is —NH—C(═O)—V, U₁ is -Q-NR₈-Q-
V is

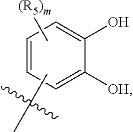

R₅ is Cl, and
m is 0 or 1.

In some embodiments, the process of preparing a compound of Formula (I) comprises contacting a compound of Formula (I′-1) with a compound of Formula (II′-1) or Formula (II′-2), wherein the compound of Formula (I) is represented by

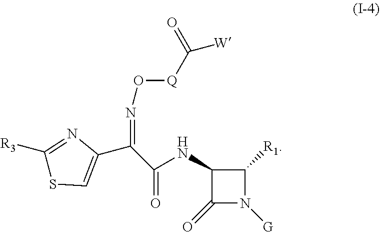

In some embodiments, Formula (I) is represented by

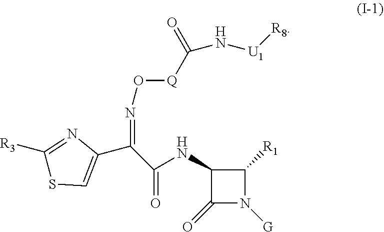

In some embodiments, Formula (I) is represented by

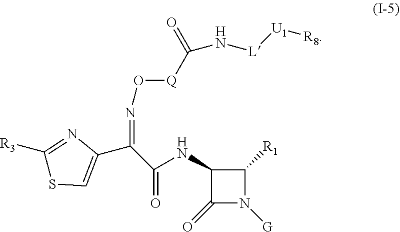

In some embodiments, the process of preparing a compound of Formula (I) comprises contacting a compound of Formula (I′-1), wherein Formula (I′-1) is represented by

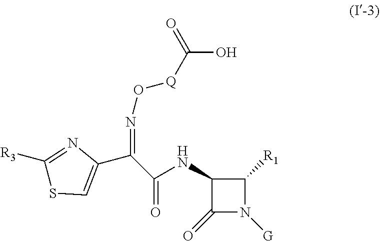

In some embodiments, the process of preparing a compound of Formula (I) comprises contacting a compound of Formula (I′-1), wherein Formula (I′-1) is represented by Formula (I′-2)

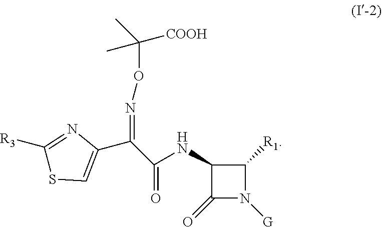

In some embodiments, Formula (I′-1) is represented by Formula (I′-3)

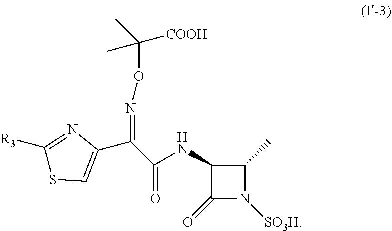

In some embodiments, Formula (II′-1) or Formula (II′-2) is selected from the group consisting of

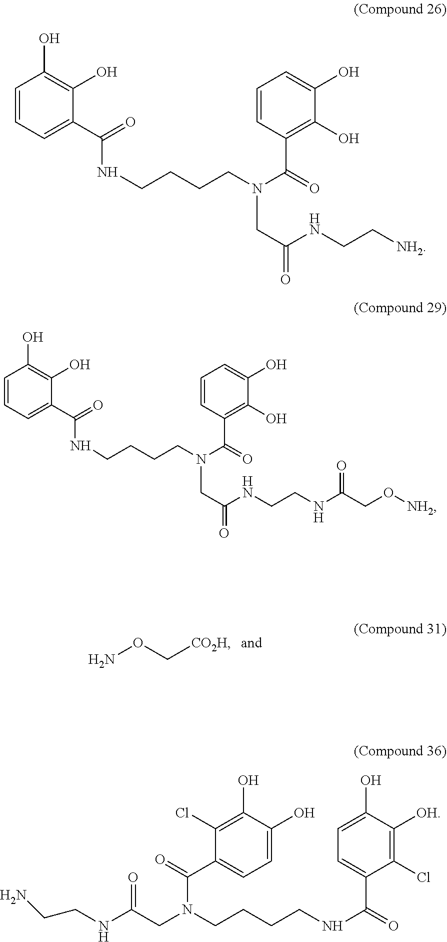 [SMILES: NCCNC(=O)CN(CCCCNC(=O)c1cccc(O)c1O)C(=O)c1cccc(O)c1O ; NOCC(=O)NCCNC(=O)CN(CCCCNC(=O)c1cccc(O)c1O)C(=O)c1cccc(O)c1O ; NOCC(=O)O ; "and" ; NCCNC(=O)CN(CCCCNC(=O)c1ccc(O)c(O)c1Cl)C(=O)c1ccc(O)c(O)c1Cl]

Although the compounds described herein may be shown with specific stereochemistry around certain atoms, such as cis or trans, the compounds can also be made in the opposite orientation or in a racemic mixture. Such isomers or racemic mixtures are encompassed by the present disclosure. Additionally, although the compounds are shown collectively in a table, any compounds, or a pharmaceutically acceptable salt thereof, can be chosen from the table and used in the embodiments provided for herein.

In some embodiments, pharmaceutical compositions comprising a compound or pharmaceutically salt thereof of any compound described herein are provided.

The compounds described herein can be made according to the methods described herein and in the examples. The methods described herein can be adapted based upon the compounds desired and described herein. In some embodiments, this method can be used to make one or more compounds as described herein and will be apparent to one of skill in the art which compounds can be made according to the methods described herein.

The conditions and temperatures can be varied, such as shown in the examples described herein. These schemes are non-limiting synthetic schemes and the synthetic routes can be modified as would be apparent to one of skill in the art reading the present specification. The compounds can also be prepared according to the schemes described in the Examples.

Although the compounds in the tables above or in the examples section are shown with specific stereochemistry around certain atoms, such as cis or trans, the compounds can also be made in the opposite orientation or in a racemic mixture.

In some embodiments, the present embodiments provide pharmaceutical compositions comprising a compound or pharmaceutically salt thereof any compound described herein.

In some embodiments, the compounds are made according to schemes described in the examples. The schemes can be used to prepare the compounds and compositions described herein.

The conditions and temperatures can be varied, or the synthesis can be performed according to the examples described herein with modifications that are readily apparent based upon the compound being synthesized.

The conditions and temperatures can be varied, such as shown in the examples described herein. These schemes are non-limiting synthetic schemes and the synthetic routes can be modified as would be apparent to one of skill in the art reading the present specification.

The present disclosure also provides the following non-limiting embodiments:

In order that the embodiments disclosed herein may be more efficiently understood, examples are provided below. It should be understood that these examples are for illustrative purposes only and are not to be construed as limiting the embodiments in any manner.

The following examples are illustrative, but not limiting, of the processes described herein. Other suitable modifications and adaptations of the variety of conditions and parameters normally encountered in therapy, synthesis, and other embodiments disclosed herein are within the spirit and scope of the embodiments.

### EXAMPLES

New conjugates (27 and 30) were synthesized in which a bis-catechol siderophore is directly attached to the side chain aminothiazoloxime (ATMO) carboxylic acid of commercially available aztreonam (9a). Another new conjugate the hydroxamic acid (32) was also synthesized derived from aminooxyacetic acid. The new conjugate, corresponding bis-catechol conjugate (30), was also synthesized. Bis-catechol antibiotic conjugates (13-15) are active indicating that the bis-catechol facilitates uptake by usually MDR resistant Gram-negative bacteria. SQ 83,280 (20) and BAL 30072 (22), without free carboxylic acids, retained potent in vitro activity. Hydroxamic acids (pKa ˜8.5-9.5) are not as acidic as carboxylic acids. The activity of derivative 32 was compared to aztreonam and with the corresponding amide (27) and hydroxamate (30) conjugates. The syntheses provide straightforward and advantageous routes which only required coupling of readily available derivatives (26 and 29) of siderophore mimetics to commercially available aztreonam (9a).

In some embodiments, examples of the siderophore moiety include, but are not limited to those shown below and as further described in Hider, R. C., and Kong, X. L. Chemistry and biology of siderophores. Nat. Prod. Rep. 2010, 27, 637-657.

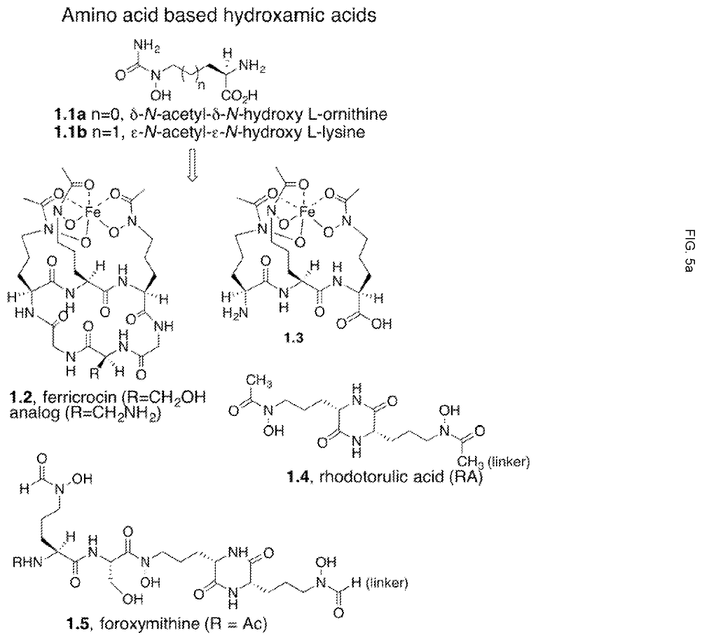

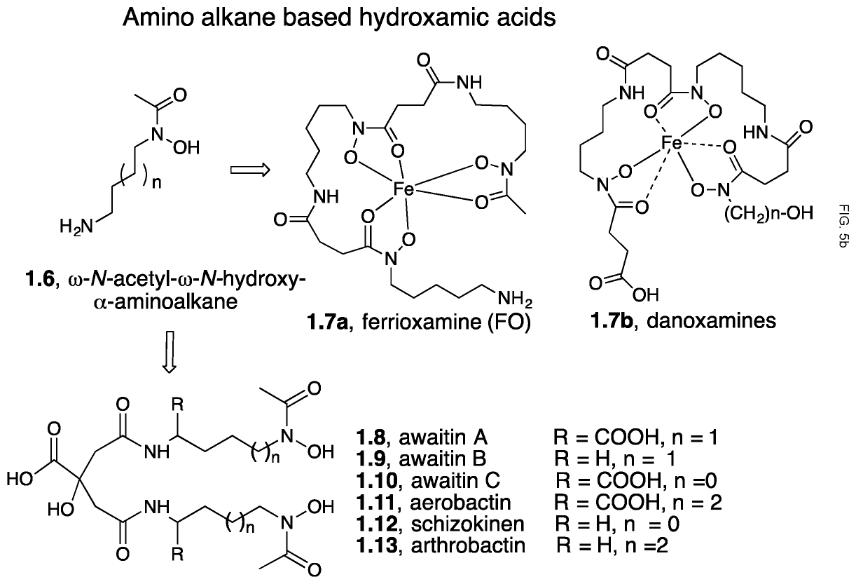

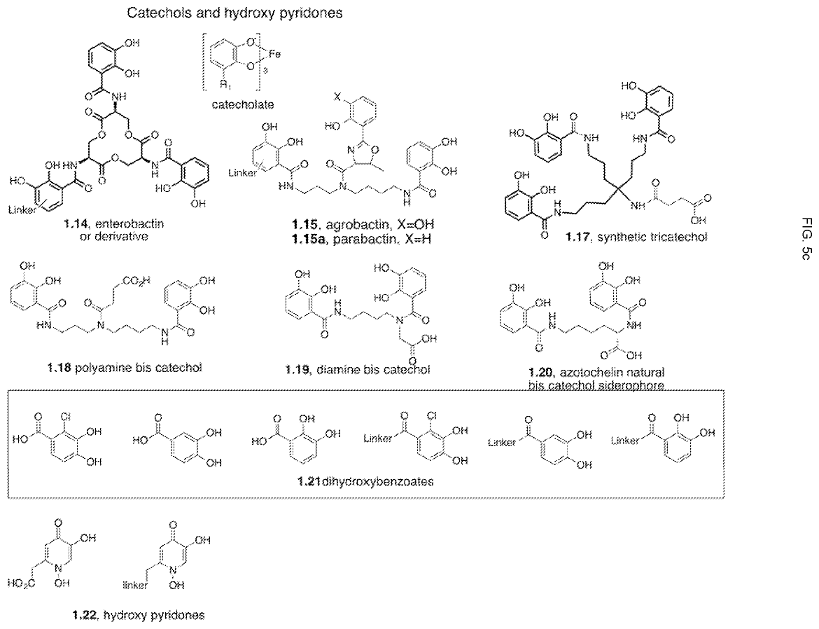

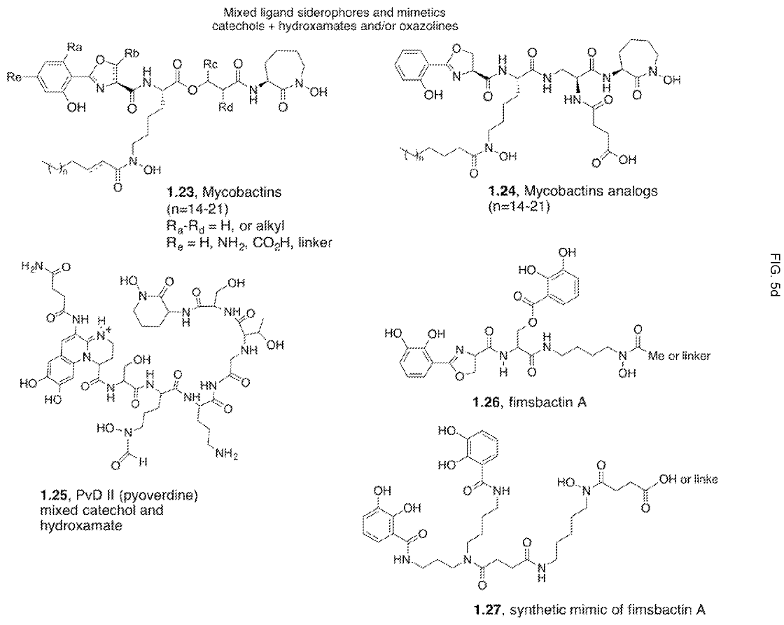

Representative siderophores, analogs and mimetics (FIG. 5a-d) include but are not limited to hydroxamic acids, catechols, alpha-hydroxy acids, oxazolines, oxazoles, hydroxy pyridines.

### Example 1. Synthesis of the New Conjugates

Schemes 2a and 2b provide synthesis routes for both dihydroxybenzoate bis catechol conjugates and chloro dihydroxybenzoate bis catechol conjugates respectively. For the dihydroxy benzoate conjugates, the chemistry started with reaction of ethane 1,2-diamine with CbzCl to give mono-Cbz protected diamine 24. Formation of the active ester of tetrabenzyl bis-catechol (25) provided the siderophore derivative (26) with an amine suitable for coupling with aztreonam (9a) to give conjugate 27. Alternatively, prior coupling of siderophore amine 26 with Boc-protected aminooxyacetic acid (28) gave hydroxylamine 29. Treatment of an aqueous solution of hydroxylamine 29 and aztreonam (9a) with EDC at pH 4.5 in aqueous THF provided the final conjugate, 30, with the hydroxamate linkage. The control aminooxyacetic acid derivative 32 was prepared by EDC/NHS activation of aztreonam followed by reaction with aminooxyacetic acid (31). For the chloro dihydroxybenzoate conjugate, the protected bis catechol methyl ester 33 was first saponified to generate free acid 34. The free acid was coupled to diamine 24 to give the protected chloro dihydroxybenzoate with the Cbz protected linker (35). Hydrogenolytic global deprotection produced free amine 36 that was then coupled to aztreonam (9a) to give the final conjugate (37).

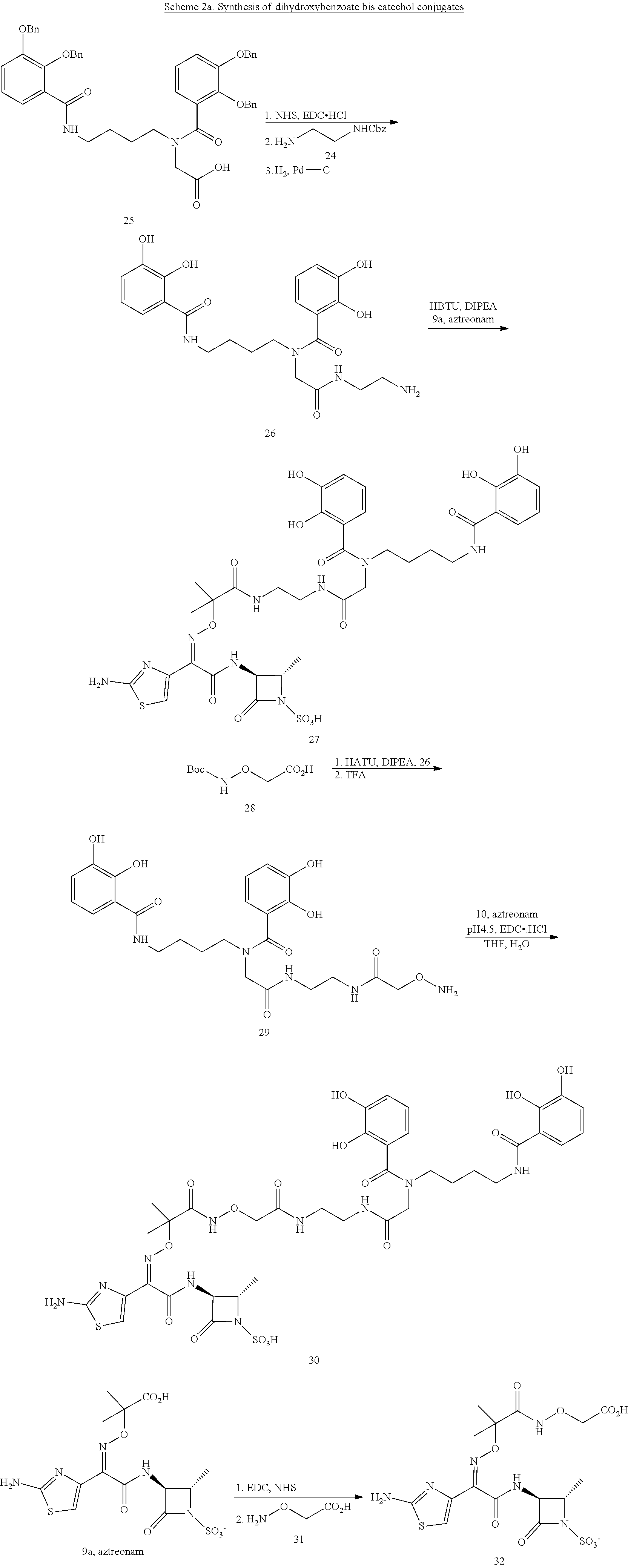

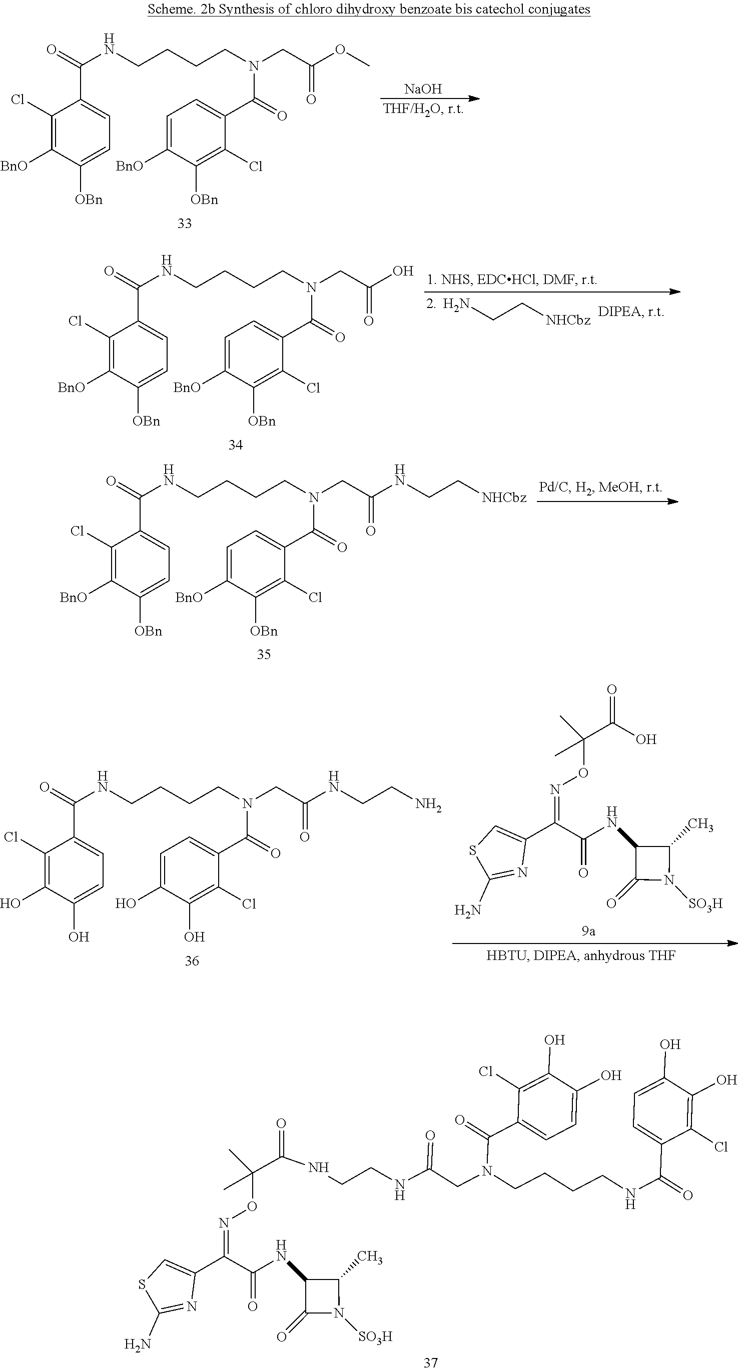

### Example 2. Antibacterial Assays

Antibacterial assay results of new aztreonam derivatives (27, 30, 32 and 37), aztreonam (9a) and earlier data of conjugate 23 are shown in Table 1. The assays were performed using iron deficient media appropriate for screening siderophore conjugates as described previously. Aminooxyacetic acid derivative 32 displayed activity comparable to that of aztreonam (9a) itself. As expected, none of the compounds were active against the Gram-positive S. aureus. However, the conjugates (27, 30 and 37) were notably and unexpectedly active against Gram-negative bacteria that are resistant to aztreonam. Of special interest is the significant inhibitory activity against problematic strains of A. baumannii and P. aeruginosa, including cephalosporinase and carbapenemase producers (A. baumannii TCC 17978 pNT320 and ATCC 17978 pNT165, respectively) which are on the World Health Organization (WHO) list of multi-drug resistant (MDR) pathogens of greatest concern. Remarkably, the activity of the new conjugates (27, 30 and 37) was also comparable to or better than that of the previously described more synthetically complex bis-catechol conjugate 23.

TABLE 1
Results of duplicate in vitro antibacterial assays of new compounds 27, 30, 32 & 37
and comparison to aztreonam (9a) and bis-catechol monobactam derivative 23.
MIC in μM

| | 27 | 30 | 32 | Aztreonam 9a | 23 | 37 |
|---|---|---|---|---|---|---|
| S. aureus | >25 | >25 | >25 | >25 | >50 | nt |
| SG511 | | | | | | |
| A. | 0.8 | 0.8 | >25 | >25 | 0.2 | 0.8 |
| baumannii | | | | | | |
| ATCC 17961 | | | | | | |
| A. | 3 | 1.6 | >25 | >25 | 0.4 | 0.8 |
| baumannii | | | | | | |
| ATCC BAA | | | | | | |
| 1797 | | | | | | |
| A. | 1.6/0.8 | 3/1.6 | >25 | >25/>25 | 0.4 | 0.4 |
| baumannii | | | | | | |
| ATCC 17978 | | | | | | |
| pNT320a | | | | | | |
| A. | 6/6.25 | 6.25/3 | nt | >25>25 | 0.4 | 25 |
| baumannii | | | | | | |
| ATCC 17978 | | | | | | |
| pNT165b | | | | | | |
| B. dolosa | >25/(>25) | >25/(>25) | >25/(>25) | >25/(>25) | >50 | nt |
| AU0018 | | | | | | |
| P. | 0.8/0.6 | 0.8/0.4 | nt | 25/6 | 0.4 | 1.2 |
| aeruginosa | | | | | | |
| 01 | | | | | | |
| P. | 0.2/0.8 | 0.8/0.4 | 25 | 12.5/3/3 | 0.4 | 0.2 |
| aeruginosa | | | | | | |
| KW799/wt | | | | | | |
| P. | 0.8/(1.6)/1.6 | 3/(0.8)/3 | >25/(>25) | >25/(>25)>25 | >50 | 4.5 |
| aeruginosa | | | | | | |
| ARC 3502 | | | | | | |
| P. | 0.8/(0.4)/0.8 | 0.8(0.8)/0.8 | >25/(>25) | >25/(6)/(25)/>25 | 1.6 | 0.4 |
| aeruginosa | | | | | | |
| ISR14-003 | | | | | | |
| P. | 0.4/(0.4)/0.2 | 0.4(0.8)/0.1 | >25/(25) | 12.5/(6)/(6)/6 | 6 | 0.2 |
| aeruginosa | | | | | | |
| ISR14-004 | | | | | | |
| E. coli DCO | 0.025 | 0.025 | 1.6 | 0.8/0.2 | <0.025 | 0.4 |
| E. | 0.05 | 0.05 | 3 | 1.6/0.8 | 0.075 | 0.8 |
| aerogenes | | | | | | |
| X816 | | | | | | |
| P. mirabilis | <0.013 | <0.013 | 0.05 | 0.05/<0.013 | <0.025 | <0.025 |
| X235 | | | | | | |
| C. freundii | 0.4 + PU | 0.4 + PU | 3 | 1.6/0.4 | 0.05 | <0.025 |
| ATCC 19063 | | | | | | PU |
| K. | 0.05 | 0.05 | 0.8 | 0.2 | nt | 0.2 |
| pneumoniae | | | | | | |
| ATCC 8303 | | | | | | |
| X68 | | | | | | |

^{a}cephalosporinase producing strain
^{b}carbapenemase producing strain
NT = not tested

The above examples demonstrated that direct coupling of siderophore like compounds to the free carboxylic acid of the commercially available monobactam, aztreonam (9a). The invention provides rapid access to conjugates with significantly enhanced antibacterial activity, including against MDR strains.

### Example 3. Detailed Description of Syntheses

### 3.1 Methods and Materials

All solvents and reagents were obtained from commercial sources and used without further purification unless otherwise stated. Silica gel (230-400 mesh) was purchased from Silicycle, Quebec City, Canada. All compounds are >98% pure by HPLC analysis. All compounds were analyzed for purity by HPLC and characterized by ¹H and ¹³C NMR using Bruker 500 MHz NMR spectrometer. The mass spectra values are reported as m/z, and HRMS analyses were carried out with a Bruker MicroOTOF-Q II, electrospray ionization time-of-flight mass spectrometer. The liquid chromatography mass spectrum (LC/MS) analyses were carried out on a Waters ZQ instrument consisting of chromatography module Alliance HT, photodiode array detector 2996, and mass spectrometer Micromass ZQ, using a 3×50 mm Pro C18 YMC reverse phase column. Mobile phases: 10 mM ammonium acetate in HPLC grade water (A) and HPLC grade acetonitrile (B). A gradient was formed from 5% to 80% of B in 10 min at 0.7 mL/min. The MS electrospray source operated at capillary voltage 3.5 kV and a desolvation temperature of 300° C.

3.2 Benzyl (2-aminoethyl)carbamate (24)

A solution of benzyl chloroformate (1.3 mL, 9 mmol) in dry DCM (25 mL) was added over 1.5 h to a solution of ethylenediamine (6 mL, 90 mmol) in dry DCM (90 mL) at 0° C. under an argon atmosphere. The mixture was stirred at 0° C. for 2 h, and then washed with brine (30 mL×3 times). The DCM layer was dried with Na₂SO₄, and concentrated under reduced pressure. Compound 24 was obtained as a white solid that was used directly for the next step without purification.

3.3 N-(2-((2-aminoethyl)amino)-2-oxoethyl)-N-(4-(2,3-dihydroxybenzamido)butyl)-2,3-di-hydroxybenzamide (26)

To a solution of compound 25 (1 mmol, 779 mg) in 10 mL of dry DMF was added N-hydroxysuccinimide (1.5 mmol, 172 mg) and EDC·HCl (2 mmol, 382 mg). The mixture was stirred at room temperature and monitored by TLC. When the starting material 25 was completely consumed, compound 24 (1.2 mmol, 233 mg) was added to the reaction mixture, and stirred for several hours and monitored by LC/MS. When the reaction was completed, the solution was diluted with H₂O and extracted with EtOAc (30 mL×3 times). The EtOAc layers were combined and dried with Na₂SO₄, concentrated under reduced pressure evaporation. The residue was purified on silica gel column chromatography eluting with DCM and 2-propanol (100:3) to give benzyl (2-(2-(2,3-bis(benzyloxy)-N-(4-(2,3-bis(benzyloxy)benzamido)butyl)benzamido)acetami do)ethyl)carbamate as colorless oil in 79% yield (753 mg, 0.79 mmol). ¹H-NMR (MeOD, 500 MHz): δ 1.03-1.04 (m, 5H), 3.61-4.21 (m, 2H), 2.95-3.15 (m, 7H), 4.82-4.87 (m, 1H), 4.94 (s, 1H), 5.00-5.02 (m, 3H), 5.05 (d, 1H, J=5.0 Hz), 5.07 (d, 1H, J=5.0 Hz), 5.10-5.15 (m, 3H), 7.04-7.46 (m, 31H).

To a solution of benzyl (2-(2-(2,3-bis(benzyloxy)-N-(4-(2,3-bis(benzyloxy) benzamido)butyl) benzamido)acetamido)ethyl)carbamate (0.5 mmol, 477 mg) in 15 mL of MeOH under an argon atmosphere was added 10% Pd/C (47 mg, 10% wt. of the oil). The reaction flask was degassed and then refilled with H₂ by a balloon. After stirred overnight, the reaction was completed, so the reaction was filtered and the filtrate was concentrated under reduced pressure to give compound 26 was as a light-purple solid in 76% yield that was used directly for the next step without purification.

3.4 (2S,3S)-3-((Z)-18-(2-Aminothiazol-4-yl)-7-(2,3-dihydroxybenzoyl)-1-(2,3-dihydroxy-phenyl)-15,15-dimethyl-1,9,14-trioxo-16-oxa-2,7,10,13,17-pentaazanonadec-17-en-19-amido)-2-methyl-4-oxoazetidine-1-sulfonic acid (27)

To the solution of aztreonam (9a, 0.5 mmol, 217 mg) in 10 mL of DMF was added HBTU (0.76 mmol, 288 mg) and DIPEA (2 mmol, 348 μL). The mixture was stirred for 10 min at room temperature. Then compound 26 (0.5 mmol, 230 mg) in 2 mL of DMF was added to the above reaction mixture. The solution was stirred overnight and monitored by LC/MS. When the reaction was completed, the solvent was removed under reduced pressure evaporation and the residue was purified by prep-HPLC to give compound 27 as a white solid in 18.3% yield (80 mg, 0.09 mmol).

¹H-NMR (DMSO-d₆, 500 MHz): δ 1.11-1.42 (m, 12H), 1.54 (brs, 1H), 3.03-3.29 (m, 8H), 3.66-3.72 (m, 2H), 3.95-4.02 (m, 1H), 4.50 (s, 1H), 6.49-6.65 (m, 3H), 6.72-6.69 (m, 2H), 6.87 (d, 1H, J=5 Hz), 6.96 (s, 1H), 7.07 (s, 1H), 7.17-7.22 (m, 1H), 7.29-7.37 (m, 3H), 7.88 (t, 1H, J=5 Hz), 8.66-8.78 (m, 1H), 9.09-9.13 (m, 1H), 9.32-9.35 (m, 1H), 9.41-9.46 (m, 1H), 12.80-12.90 (m, 1H). ¹³C-NMR (DMSO-d₆, 500 MHz): δ 18.67, 24.60, 24.96, 26.03, 26.47, 38.79, 39.12, 48.11, 49.45, 51.45, 57.71, 60.94, 83.61, 111.20, 115.52, 116.22, 116.66, 117.74, 118.19, 118.53, 119.42, 119.96, 120.19, 125.08, 141.84, 142.94, 145.90, 146.85, 150.40, 150.99, 162.85, 163.30, 169.47, 169.92, 170.37, 174.36. HRMS calcd for C₃₅H₄₄N₉O₁₄S₂(M+H⁺) 878.2444; found 878.2429.

3.5 2-(((tert-Butoxycarbonyl)amino)oxy)acetic acid (28)

A round-bottom flask was charged with carboxymethoxylamine hemihydrochloride (31, 2 mmol, 220 mg) in 15 mL of dry DCM. The solution was cooled to 0° C., and Et₃N (6 mmol, 424 μL) was added. To the mixture was added a solution of (Boc)₂O (3 mmol, 1.3 g) in 10 mL of DCM. The reaction was stirred at 0° C. for 30 min, then warmed up to room temperature. When the reaction was completed, the solution was washed with water. The aqueous portion was extracted with 20 mL of EtOAc and this EtOAc was discarded. Then the pH value of the aqueous portion was adjusted to 3.5 with 1N HCl, and extracted with EtOAc (30 mL×3 times). The EtOAc layers were combined and concentrated under reduced pressure to give compound 28 as a white solid, which used directly without purification.

3.6 N-(2-((2-(2-(Aminooxy)acetamido)ethyl)amino)-2-oxoethyl)-N-(4-(2,3-dihydroxy-benzamido) butyl)-2,3-dihydroxybenzamide (29)

To the solution of 28 (0.98 mmol, 187 mg) in DMF (5 mL) was added HBTU (1.34 mmol, 505 mg) and DIPEA (3.56 mmol, 656 μL), the mixture was stirred 10 min at room temperature. Then compound 26 (409 mg, 0.89 mmol) in DMF (2 mL) was added to the above solution and the reaction was continued overnight. When the reaction was completed, the solvent was removed by reduced pressure evaporation, and the residue was purified on silica gel column chromatography eluting with DCM and MeOH (20:1) to give tert-butyl ((7-(2,3-dihydroxybenzoyl)-1-(2,3-dihydroxyphenyl)-1,9,14-trioxo-2,7,10,13-tetraazapentadecan-15-yl)oxy)carbamate as alight yellow solid in 45% yield (170 mg, 0.27 mmol). ¹H-NMR (MeOD, 500 MHz): δ 1.41-1.50 (m, 11H), 1.57-1.60 (m, 1H), 1.73 (brs, 1H), 3.24 (t, 2H, J=5 Hz), 3.35-3.61 (m, 6H), 3.98-4.25 (m, 4H), 6.69-6.72 (m, 3H), 6.83-6.85 (m, 1H), 6.91 (dd, 1H, J₁=5 Hz, J₂=10 Hz), 7.16-7.22 (m, 1H).

tert-butyl ((7-(2,3-dihydroxybenzoyl)-1-(2,3-dihydroxyphenyl)-1,9,14-trioxo-2,7,10,13-tetraazapentadecan-15-yl)oxy)carbamate (80 mg, 0.13 mmol) was dissolved in 15 mL of dry DCM followed by adding 1 mL of TFA. The mixture was stirred at room temperature and monitored by LC/MS. When the reaction was completed, the reaction was concentrated under reduced pressure evaporation to give compound 29 that was used directly for the next step without further purification.

3.7 (2S,3S)-3-((Z)-22-(2-Aminothiazol-4-yl)-7-(2,3-dihydroxybenzoyl)-1-(2,3-dihydroxyphenyl)-19,19-dimethyl-1,9,14,18-tetraoxo-16,20-dioxa-2,7,10,13,17,21-hexaazatricos-21-en-23-amido)-2-methyl-4-oxoazetidine-1-sulfonic acid (30)

Compound 29 (67 mg, 0.12 mmol) and aztreonam (9a, 156 mg, 0.36 mmol) were dissolved in H₂O/THF (2 mL/2 mL) to give a solution. The pH value of the solution was adjusted to 4.5 by adding NaOH (1N). EDC·HCl (46 mg, 0.24 mmol) was dissolved in 2 mL of H₂O and then slowly added to the above mixture while keeping pH value at 4.5 by adding HCl (1N). When the pH did not change during EDC·HCl adding, the reaction was completed and confirmed by LC/MS. The result solution was purified by prep-HPLC directly without workup. Compound 30 was obtained as a white solid in 35% yield (40 mg, 0.04 mmol). ¹H-NMR (DMSO-d₆, 500 MHz): δ 1.30 (brs, 1H), 1.36 (brs, 6H), 1.40-1.43 (m, 4H), 1.55 (brs, 2H), 3.08 (brs, 4H), 3.17 (brs, 4H), 3.66-3.70 (m, 1H), 3.73 (s, 1H), 4.03 (s, 1H), 4.22-4.25 (m, 2H), 4.49 (dd, 1H, J₁=5 Hz, J₂=10 Hz), 6.50-6.64 (m, 3H), 6.70-6.77 (m, 1H), 6.80 (s, 1H), 6.87 (d, 1H, J=5 Hz), 6.96 (s, 1H), 7.06 (s, 1H), 7.16 (s, 1H), 7.20-7.29 (m, 1H), 7.32 (s, 1H), 7.92 (s, 1H), 8.23 (d, 1H, J=20 Hz), 8.66-8.78 (m, 1H), 9.09 (d, 1H, J=20 Hz), 9.23 (d, 1H, J=10 Hz), 9.48 (d, 1H, J=10 Hz), 11.00 (s, 1H), 12,79-12.89 (m, 1H). ¹³C-NMR (DMSO-d₆, 500 MHz): δ 17.13, 23.33, 23.52, 25.68, 26.14, 26.38, 28.34, 38.76, 48.49, 49.92, 58.24, 61.42, 75.13, 83.75, 111.89, 115.52, 116.33, 117.50, 117.82, 118.38, 120.04, 120.29, 123.35, 141.08, 141.53, 145.41, 145.68, 146.11, 149.13, 149.99, 163.73, 163.88, 169.97, 170.35, 170.48, 171.77, 172.86. HRMS calcd for C₃₇H₄₇N₁₀O₁₆S₂ (M+H⁺) 951.2607; found 951.2611.

3.8 (Z)-2-(2-Aminothiazol-4-yl)-5,5-dimethyl-1-(((2S,3S)-2-methyl-4-oxo-1-sulfoazetidin-3-yl)amino)-1,6-dioxo-4,8-dioxa-3,7-diazadec-2-en-10-oic acid DIPEA salt (32)

To a solution of aztreonam (9a, 0.3 mmol, 130 mg) in 4 mL of DMF was added N-hydroxysuccinimide (1.2 mmol, 138 mg) and EDC·HCl (1.5 mmol, 287 mg). The reaction was stirred at room temperature and monitored by LC/MS. When the aztreonam was completely converted to the NHS active ester indicated by the LC/MS, carboxymethoxylamine hemihydrochloride (31, 3 mmol, 327 mg) was added to the reaction solution, followed by adding DIPEA (6 mmol, 1.1 mL). The reaction was continue stirred at room temperature for several hours and monitored by LC/MS. When the reaction was completed, the solution was concentrated by reduced pressure evaporation, and the residue was purified by prep-HPLC. Compound 32 was obtained as colorless oil in 75% yield (115 mg, 0.23 mmol). ¹H-NMR (MeOD, 500 MHz): δ 1.34-1.37 (m, 15H), 1.53 (s, 3H), 1.54 (s, 3H), 1.59 (s, 3H), 3.19 (q, 2H, J=5 Hz), 3.69-3.74 (m, 2H), 4.13-4.15 (m, 1H), 4.22 (d, 2H, J=5 Hz), 4.55 (d, 1H, J=5 Hz), 6.95 (s, 1H). ¹³C-NMR (MeOD, 500 MHz): δ 12.00, 16.68, 17.10, 22.58, 23.39, 23.50, 42.52, 54.51, 57.99, 61.60, 74.11, 83.53, 111.54, 141.82, 150.34, 163.74, 164.06, 171.96, 174.77. HRMS calcd for C₁₅H₂₀N₆NaO₁₀S₂ (M+Na⁺) 531.0575; found 531.0599.

3.9 N-(4-(3,4-bis(benzyloxy)-2-chlorobenzamido)butyl)-N-(3,4-bis(benzyloxy)-2-chlorobenzoyl)glycine (34)

To a solution of 33 (2 mmol, 1.72 g) in THF/H₂O (10 mL/5 mL) was added NaOH (2.8 mmol, 112 mg). The mixture was stirred at room temperature for 3 hours. When 33 was totally consumed, 1N HCl was added to adjust the pH value to 5. The solution was extracted with EtOAc (30 mL×3 times), the organic layer was dried over Na₂SO₄, and concentrated under reduced pressure evaporation. The product 34 was obtained as a yellow solid (1.56 g, 92% yield) that used directly for the next step reaction without further purification.

3.10 Benzyl (2-(2-(3,4-bis(benzyloxy)-N-(4-(3,4-bis(benzyloxy)-2-chlorobenzamido)butyl)-2-chlorobenzamido)acetamido)ethyl)carbamate (35)

To a solution of 34 (1.84 mmol, 1.56 g) in anhydrous DMF (20 mL) was added N-hydroxysuccinimide (2.76 mmol, 317 mg) and EDC·HCl (3.68 mmol, 705 mg). The mixture was stirred at room temperature for 3 hours and monitored by LC/MS. When 34 was totally consumed, benzyl (2-aminoethyl)carbamate (2 mmol, 388 mg) and DIPEA (4 mmol, 736 mg) were added to the reaction. The mixture was stirred overnight, and monitored by LC/MS. When the reaction was completed, the solvent was concentrated by reduced pressure evaporation. The residue was purified by silica gel chromatography to give 35 as a white solid (1.53 g, 81% yield). ¹H-NMR (DMSO-d₆, 500 MHz): δ 1.24-1.27 (m, 1H), 1.44-1.55 (m, 2H), 1.57-1.61 (m, 1H), 2.97-3.09 (m, 5H), 3.13-3.23 (m, 2H), 3.52 (d, 1H, J=20 Hz), 3.72 (d, 1H, J=15 Hz), 3.86 (d, 1H, J=10 Hz), 4.93-5.00 (m, 6H), 5.16-5.22 (m, 4H), 6.99-7.49 (m, 29H), 7.90-7.96 (m, 2H), 8.21-8.34 (m, 1H).

3.11 N-(2-((2-aminoethyl)amino)-2-oxoethyl)-2-chloro-N-(4-(2-chloro-3,4-dihydroxybenzamido)butyl)-3,4-dihydroxybenzamide (36)

35 (120 mg, 0.12 mmol) in 20 mL of MeOH under an argon atmosphere was added 10% Pd/C (24 mg, 10% wt. of 35). The reaction flask was degassed and then refilled with H₂ by a balloon. After stirred overnight, the reaction was completed, so the reaction was filtered and the filtrate was concentrated under reduced pressure evaporation to give 36 was as a colorless oil that was used directly for the next step without purification.

3.12 2S,3S)-3-((Z)-18-(2-aminothiazol-4-yl)-7-(2-chloro-3,4-dihydroxybenzoyl)-1-(2-chloro-3,4-dihydroxyphenyl)-15,15-dimethyl-1,9,14-trioxo-16-oxa-2,7,10,13,17-pentaazanonadec-17-en-19-amido)-2-methyl-4-oxoazetidine-1-sulfonic acid (37)

To the solution of aztreonam (9a, 68 mg, 0.13 mmol) in 6 mL of DMF was added HBTU (0.17 mmol, 63 mg) and DIPEA (0.44 mmol, 81 μL). The mixture was stirred for 10 min at room temperature. Then 36 (0.11 mmol, 53 mg) in 2 mL of DMF was added to the above reaction mixture. The solution was stirred overnight and monitored by LC/MS. When the reaction was completed, the solvent was removed under reduced pressure evaporation and the residue was purified by prep-HPLC to give 37a as an off-white solid in 63.5% yield (66 mg, 0.069 mmol). ¹H-NMR (DMSO-d₆, 500 MHz): δ 1.19-1.23 (m, 13H), 1.33-1.54 (m, 7H), 2.90-3.17 (m, 6H), 3.57-3.63 (m, 2H), 3.67-3.72 (m, 1H), 3.83-4.06 (m, 1H), 4.43-4.52 (m, 1H), 6.49-6.80 (m, 3H), 7.09 (brs, 2H), 7.28-7.40 (m, 2H), 7.82-7.86 (m, 1H), 8.01-8.14 (m, 1H), 9.13-9.34 (m, 1H).

### REFERENCES

As various changes can be made in the below-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings. Accordingly, the present description is intended to embrace all such alternatives, modifications, and variances which fall within the scope of the appended claims. All patents, applications, publications, test methods, literature, and other materials cited herein are hereby incorporated by reference in their entirety as if physically present in this specification.
1. Sheehan, J. C. The Enchanted Ring: The Untold Story of Penicillin; MIT Press: Cambridge, MA, 1982.
2. (a) Bush, K, and P. A. Bradford. in: “Enzyme-mediated resistance to antibiotics: mechanisms, dissemination, and prospects for inhibition.” (R. A. Bonomo and M. E. Tolmasky, eds.) ASM Press; Washington, DC: (2007), pp. 67-79, β-Lactamases: historical perspectives. (b) Bush, K.; Bradford, P. A. β-Lactams and β-Lactamase Inhibitors: An Overview. Cold Spring Harb. Perspect. Med. 2016, 6, a025247
3. O'Neill J, Chair. Tackling Drug-Resistant Infections Globally: Final Report and Recommendations. London, UK: Review on Antimicrobial Resistance; 2016; p. 1-84. (b) For an update, also see: https://www.chathamhouse.org/sites/default/files/publications/research/2019-10-11-AMR-Full-Paper.pdf
4. 2020 Antibacterial agents in clinical and preclinical development: an overview and analysis. Geneva: World Health Organization; 2021. https://www.who.int/publications/i/item/9789240021303 (accessed Jun. 14, 2021).
5. (a) Mattingly, P. G.; Kerwin, J. F., Jr.; Miller, M. J. A Facile Synthesis of Substituted N-Hydroxy-2-Azetidinones. A Biogenic Type β-Lactam Synthesis. J. Am. Chem. Soc. 1979, 101, 3983-3985. (b) Miller, M. J.; Mattingly, P. G.; Morrison, M. A.; Kerwin, J. F. Synthesis of □-Lactams from Substituted Hydroxamic Acids. J. Am. Chem. Soc. 1980, 102, 7026-7032.
6. Miller, M. J. The Hydroxamate Approach to the Synthesis of β-Lactam Antibiotics. Acc. Chem. Res. 1986, 19, 49-56.
7. Woulfe, S. R.; Miller, M. J. Synthesis and Biological Activity of [(3(S)-Acylamino-2-Oxo-1-Azetidinyl)oxy] Acetic Acids. A New Class of Heteroatom Activated β-Lactam Antibiotics. J. Med. Chem. 1985, 28, 1447-1453.
8. Flanagan, M. E.; Brickner, S. J. et al Preparation, Gram-Negative Antibacterial Activity, and Hydrolytic Stability of Novel Siderophore-Conjugated Monocarbam Diols.
9. Cimarusti, C. M.; Sykes, R. B. Monocyclic D-Lactam Antibiotics. Med. Res. Rev. 1984, 4, 1-24.
10. (a) Sykes, R. B.; Cimarusti, C. M.; Bonner, D. P.; Bush, K.; Floyd, D. M.; Georgopapadakou, N. H.; Koster, W. H.; Liu, W. C.; Parker, W. L.; Principe, P. A.; Rathnum, M. L.; Slusarchyk, W. A.; Trejo, W. H.; Wells, J. S. Monocyclic β-Lactam Antibiotics Produced by Bacteria. Nature 1981, 291, 489-491. (b) Imada, A.; Kitano, K.; Kintaka, K.; Muroi, M.; Asai, M. Sulfazecin and Isosulfazecin, Novel β-Lactam Antibiotics of Bacterial Origin. Nature 1981, 289, 590-591.
11. Decuyper, L.; Jukic, M.; Sosic, I.; Zula, A.; D'hooghe, M. D.; Gobec, S. Antibacterial and β-Lactamase Inhibitory Activity of Monocyclic β-Lactams. Med. Res. Rev. 2017, 38, 426-503.
12. Sykes, R. B.; Bonner, D. P.; Bush, K.; Georgopapadakou, N. H. Azthreonam (SQ 26,776), a Synthetic Monobactam Specifically Active Against Aerobic Gram-negative Bacteria. Antimicrob. Agents Chemother. 1982, 21, 85-92.
13. McNulty C A, Garden G M, Ashby J, Wise R (September 1985). Pharmacokinetics and tissue penetration of carumonam, a new synthetic monobactam Antimicrob. Agents Chemother. 1985, 28, 425-427.
14. Blais, J.; Lopez, S.; Li, C.; Rukzin, A.; Ranjitkar, S.; Dean, C. R.; Leeds, J. A.; Casarez, A.; Simmons, R. L.; Reck, F. In Vitro Activity of LYS228, a Novel Monobactam Antibiotic, against Multidrug-Resistant Enterobacteriaceae. Antimicrob. Agents Chemother. 2018, 62, e00552-18.
15. (a) Slusarchyk, W. A.; Dejneka, T.; Gordon, E. M.; Weaver, E. R.; Koster, W. H. Monobactams: Ring Activating N-1 Substituents in Monocyclic D-Lactam Antibiotics. Heterocycles 1984, 21, 191-209. (b) Floyd, D. M.; Fritz, W.; Cimarusti, C. M. Monobactams. Stereospecific Synthesis of (S)-3-amino-2-oxoazetidine-1-sulfonic Acids. J. Org. Chem. 1982, 47, 176-178.
16. (a) Wencewicz, T. A., and Miller, M. J. (2018) Sideromycins as Pathogen-Targeted Antibiotics. In Antibacterials: Vol. II (Fisher, J. F., Mobashery, S. and Miller, M. J., Eds.), pp 151-183, Springer International Publishing. (b) Miller, M. J.; Liu, R. Design and Syntheses of New Antibiotics Inspired by Nature's Quest for Iron in an Oxidative Climate. Accts. Chem. Res. 2021, 54, 1646-1661.
17. Möllmann, U.; Heinisch, L.; Bauernfeind, A.; Kohler, T.; Ankel-Fuchs, D. Siderophores as Drug Delivery Agents: Application of the “Trojan Horse” Strategy. Biometals 2009, 22, 615-624.
18. Lin, Y.-M.; Ghosh, M.; Miller, P.; Möllmann, U.; Miller, M. J. Synthetic sideromycins (skepticism and optimizm): selective generation of either broad or narrow spectrum Gram-negative antibiotics. BioMetals 2019, 425-451.
19. Aoki, T.; Yoshizawa, H.; Yamawaki, K.; et al. Cefiderocol (S-649266), A new siderophore cephalosporin exhibiting potent activities against Pseudomonas aeruginosa and other gram-negative pathogens including multi-drug resistant bacteria: Structure activity relationship. Europ. J. Med. Chem. 2018, 155, 847-868.
20. FDA approves new antibacterial drug to treat complicated urinary tract infections as part of ongoing efforts to address antimicrobial resistance. https://www.fda.gov/news-events/press-announcements/fda-approves-new-antibacterial-drug-treat-complicated-urinary-tract-infections-part-ongoing-efforts.
21. Liu, R.; Miller, P. A.; Vakulenko, S. B.; Stewart, N. K.; Boggess, W. C.; Miller, M. J. A Synthetic Dual Drug Sideromycin Induces Gram-Negative Bacteria to Commit Suicide with a Gram-Positive Antibiotic. J. Med. Chem. 2018, 61, 3845-3854.
22. Ghosh, M.; Miller, P. A.; Möllmann, U.; Claypool, W. D.; Schroeder, V. A.; Wolter, W. R.; Suckow, M.; Yu, H.; Li, S.; Huang, W.; Zajicek, J.; Miller, M. J. Targeted Antibiotic Delivery: Selective Siderophore Conjugation with Daptomycin Confers Potent Activity Against Multi-Drug Resistant Acinetobacter baumannii Both in vitro and in vivo. J. Med. Chem. 2017, 60, 4577-4583.
23. Barbachyn, M. R.; Touminen, T. C. Synthesis and Structure-Activity Relationships of Monocarbams Leading to U-78608. J. Antibiot. 1990, 43, 1199-1203.
24. Sykes, R. B.; Koster, W. H.; Bonner, D. P. The New Monobactams: Chemistry and Biology. J. Clin. Pharmacol. 1988, 28, 113-119.
25. Flanagan, M. E.; Brickner, S. J.; Lall, M.; Casavant, J.; Deschenes, L.; Finegan, S. M.; George, D. M.; Granskog, K.; Hardink, J. R.; Huband, M. D.; Hoang, T.; Lamb, L.; Marra, A.; Mitton-Fry, M.; Mueller, J. P.; Mullins, L. M.; Noe, M. C.; O'Donnell, J. P. Pattavina, D.; Penzien, J. B.; Schuff, B. P.; Sun, J.; Whipple, D. A.; Young, J.; Gootz, T. D. Preparation, Gram-Negative Antibacterial Activity, and Hydrolytic Stability of Novel Siderophore-Conjugated Monocarbam Diols. ACS Med. Chem. Lett. 2011, 2, 385-390.
26. Page, M. G. P.; Dantier, C.; Desarbre, E. In Vitro Properties of BAL30072, a Novel Siderophore Sulfactam with Activity against Multiresistant Gram-Negative Bacilli. Antimicrob. Agents Chemother. 2010, 54, 2291-2302.
27. Tan, L.; Tao, Y.; Wang, T.; Zou, F.; Zhang, S.; Kou, Q.; Niu, A.; Chen, Q.; Chu, W.; Chen, X.; Wang, H.; Yang, Y. Discovery of Novel Pyridone-Conjugated Monosulfactams as Potent and Broad-Spectrum Antibiotics for Multidrug-Resistant Gram-Negative Infections. J. Med. Chem. 2017, 60, 2669-2684.
28. Cilibrizzi, A.; Abbate, V.; Chen, Y.-U.; Ma, Y.; Zhou, T.; Hider, R. C. Hydroxypyridone Journey into Metal Chelation. Chem. Rev. 2018, 118, 7657-7701.
29. Hider, R. C., and Kong, X. Chemistry and Biology of Siderophores. Nat. Prod. Rep. 2010, 27, 637-657.
30. Carosso, S.; Liu, R.; Miller, P. A.; Hecker, S. J.; Glinka, T.; Miller, M. J. Methodology for Monobactam Diversification: Synthesesand Studies of 4-Thiomethyl Substituted β-Lactams with Activity Against Gram-Negative Bacteria, Including Carbapenemase Producing Acinetobacter baumannii. J. Med. Chem. 2017, 60, 8933-8944.
31. Ghosh, M., Miller, P. A., Möllmann, U., Claypool, W. D., Schroeder, V. A., Wolter, W. R., Suckow, M., Yu, H., Li, S., Huang, W., Zajicek, J., and Miller, M. J. Targeted Antibiotic Delivery: Selective Siderophore Conjugation with Daptomycin Confers Potent Activity Against Multi-Drug Resistant Acinetobacter baumannii Both in vitro and in vivo,” J. Med. Chem. 2017, 60, 4577-4583.
32. Lin, Y.-M.; Ghosh, M.; Miller, P.; Möllmann, U.; Miller, M. J. Synthetic sideromycins (skepticism and optimism): selective generation of either broad or narrow spectrum Gram-negative antibiotics. Biometals, 2019, 32, 425-451.
33. Ghosh, M.; Miller, P. A.; Miller, M. J. Antibiotic repurposing: bis-catechol- and mixed ligand (bis-catechol-mono-hydroxamate)-teicoplanin conjugates are active against multidrug resistant Acinetobacter baumannii. J. Antibiot. 2020, 73, 152-157.
34. Vertesy, L.; Aretz, W.; Fehlhaber, H.-W.; Kogler, H. Salmycin A-D, antibiotika aus Streptomyces violaceus, DSM 8286, mit siderophor-aminoglycosid-struktur. Helv. Chim. Acta 1995, 78, 46-60.
35. (a) Gause, G. F. Recent studies on albomycin, a new antibiotic. Br. J. Med. 1955, 2, 1177-1179. (b) Gamburg, R. L. Use of albomycin in pneumonia in children. Pediatriia 1951, 5, 37-44.

What is claimed is:

1. A compound of Formula (I): 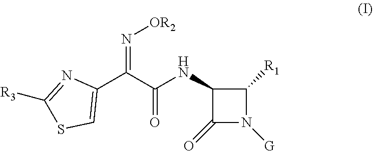
or a pharmaceutically acceptable salt or zwitterion thereof, wherein
G is —OR^{A}, —OCH₂C(═O)X, OCHRC(═O)X, —C(═O)X, —S(═O)₂X, —OS(═O)₂X, —C(═O)NH—S(═O)₂G′, P(═O)X or —P(═O)X,
G′ is OR^{A}, —OCH₂C(═O)X, OCHRC(═O)X, —C(═O)X, —S(═O)₂X, —OS(═O)₂X, P(═O)X or —P(═O)X,
X is H, —OR^{A} or —N(R^{A})₂,
R₁ is H, or —(C₁-C₁₂)alkyl optionally substituted by X or Z,
Z is R^{A}, OR^{A}, —SR^{A}, —C(═O)N(R^{A})₂, or —N(R^{A})₂,
R₂ is —CH₂C(═O)W, -Q-C(═O)W, —CHR⁴C(═O)W, —C(R⁴)₂C(═O)W, —C(R⁴)₂C(═O)NHOCH₂C(═O)W, -Q-C(═O)NHO-Q-C(═O)W, -Q-C(═O)NHO-Q-C(═O)OH, —C(R⁴)₂C(═O)NHOCH₂C(═O)OH, —C(R⁴)₂C(═O)NHOC(R⁴)₂C(═O)OH, or —C(R⁴)₂C(═O)NHOC(R⁴)₂C(═O)W,
R₃ is H, Z, or R⁴,
each R⁴ is independently H, Z, —(C₁-C₁₂)alkyl, —(C₃-C₈)cycloalkyl, —(C₃-C₆)heterocycloalkyl, or —(C₁-C₈)N(R^{A})R^{B}, or two R⁴ are taken together with the carbon atom to which both are attached to form a —(C₃-C₈)cycloalkyl or —(C₃-C₆)heterocycloalkyl,
each R^{A} is independently H, —(C₁-C₁₂)alkyl, —(C₃-C₈)cycloalkyl, phenyl, aryl, or heteroaryl,
R^{B} is H, —(C₁-C₁₂)alkyl, —(C₃-C₈)cycloalkyl, or phenyl,
W is Sid or linker-Sid,
Sid is a siderophore moiety,
linker is —NH-Q-C(═O)O—, —NH—C(═O)-Q-C(═O)O—, —C(═O)-Q-, —C(═O)O-Q-, —C(═O)-Q-C(═O)—NH—, —O-Q-O—, —O-Q-, —NH-Q-, —NH-Q-NH—, —NH-Q-O—, —NH—O-Q-, or the combination thereof,
Each Q is independently a —(C₁-C₁₂)alkylene optionally substituted by one or more selected from the group consisting of —OH, —COOH, ═O, —NH₂, —(C₁-C₁₂)alkyl, —(C₁-C₁₂)alkenyl, —(C₁-C₁₂)alkylene —OH, —(C₁-C₁₂)alkylene —NH₂, —NH—(C₁-C₁₂)alkylene-COOH, —NH—C(═O)—(C₁-C₁₂)alkylene —COOH, —C(═O)—(C₁-C₁₂)alkylene —C(═O)—NH₂, —O—(C₁-C₁₂)alkylene-OH, —NH—(C₁-C₁₂)alkylene —NH₂, and —NH—(C₁-C₁₂)alkylene —OH.

2. The compound of claim 1, or a pharmaceutically acceptable salt or zwitterion thereof, wherein Sid-OH, Sid-NH₂, Sid-COOH, or -linker-Sid is represented by Formula (II):
R₈—U₁-R⁹ (II)
wherein
U₁ is a covalent bond, or optionally substituted -Q-, —C(═O)—, —O—C(═O)—, —CHR₈—, —CR₁₀R₈—, —C(═O)—NH—, —C(═O)—NR₈—, —C(═O)—N(OH)—, —NH—, —NR₈—, —NR₁₀—, —NH—CHR₈—C(═O)—, —R₁₁—, or the combination thereof,
each R₈ is independently H, —OH, —COOH, —NH₂, -Q-OH, —V, -Q-V, —NH—C(═O)—V, -Q-NH—C(═O)—V, —NR₁₀—C(═O)—V, —NR₁₀—V, —C(═O)—V, —O—C(═O)—V, —C(═O)—R₁₁—V, —NH—C(═O)—R¹¹—V, —C(═O)—R₁₁-aryl, or linker-H,
R₉ is H, —OH, —COOH, —NH₂, -Q-OH, —V, -Q-V, —NH—C(═O)—V, -Q-NH—C(═O)—V, —NR₁₀—C(═O)—V, —NR₁₀—V, —C(═O)—V, —O—C(═O)—V, —C(═O)—R₁₁—V, —NH—C(═O)—R₁₁—V, —C(═O)—R₁₁-aryl, or linker-H, or R₈ and R₉ are taken together as a covalent bond to form a ring,
R₁₀ is —NH—C(O)-Q-COOH, —C(O)-Q-COOH, -Q-COOH, or —COOH,
R₁₁ is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S,
V═—N(OH)—C(═O)—NH₂, —N(OH)—C(═O)H, —N(OH)—C(═O)-linker, —N(OH)—C(═O)—(C₁-C₁₂)alkyl, —N(OH)—C(═O)—(C₁-C₁₂)alkenyl, —N(OH)—C(═O)-Q-OH, —N(OH)—C(═O)-Q-COOH, 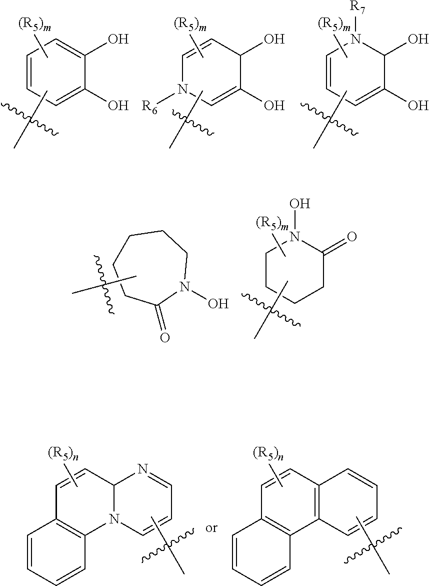
m is an integral selected from 0-3,
n is an integral selected from 0-10,
each R₅ is independently —(C₁-C₁₂)alkyl, halogen, —OH, —COOH, —NH₂, -linker-H, or —C═) linker-H, and
R₆ and R₇ are independently H, —COOH, or —OR^{A}.

3. The compound of any one of claims 1-2, or a pharmaceutically acceptable salt or zwitterion thereof, wherein U₁ is
—U₁₁—U₁₂—U₁₃—U₁₄—U₁₅—U₁₆— (II-1)
wherein
U₁₁, U₁₂, U₁₃, U₁₄, U₁₅, and U₁₆ are independently a covalent bond, or optionally substituted -Q-, —C(═O)—, —O—C(═O)—, —CHR₈—, —CR₁₀R₈—, —C(═O)—NH—, —C(═O)—NR₈—, —C(═O)—N(OH)—, —NH—, —NR₈—, —NR₁₀—, —NH—CHR₈—C(═O)—, or —R₁₁—.

4. The compound of any one of claims 1-3, or a pharmaceutically acceptable salt or zwitterion thereof, wherein G is O—CH₂—COOH, S(═O)₂OH, OS(═O)₂OH, or C(═O)NH—S(═O)₂G′.

5. The compound of any one of claims 1-4, or a pharmaceutically acceptable salt or zwitterion thereof, wherein G is S(═O)₂OH.

6. The compound of any one of claims 1-5, or a pharmaceutically acceptable salt or zwitterion thereof, wherein R₁ is —(C₁-C₁₂)alkyl.

7. The compound of any one of claims 1-6, or a pharmaceutically acceptable salt or zwitterion thereof, wherein R₁ is —CH₃.

8. The compound of any one of claims 1-7, wherein R₂ is —C(R⁴)₂C(═O)W, C(R⁴)₂C(═O)NHOCH₂C(═O)OH, or —C(R⁴)₂C(═O)NHOC(R⁴)₂C(═O)W.

9. The compound of any one of claims 1-8, or a pharmaceutically acceptable salt or zwitterion thereof, R⁴ is —CH₃.

10. The compound of any one of claims 1-9, or a pharmaceutically acceptable salt or zwitterion thereof, wherein R₃ is —NH₂.

11. The compound of any one of claims 1-10, or a pharmaceutically acceptable salt or zwitterion thereof, wherein Formula (I) is represented by

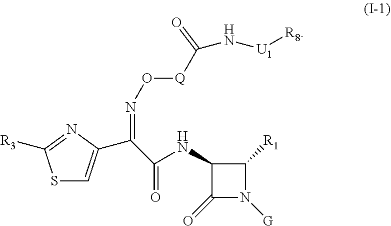

12. The compound of any one of claims 1-11, or a pharmaceutically acceptable salt or zwitterion thereof, wherein Formula (I) is represented by

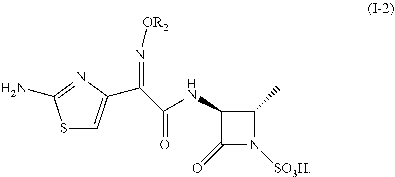

13. The compound of any one of claims 1-12, or a pharmaceutically acceptable salt or
zwitterion thereof, wherein V is

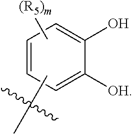

14. The compound of any one of claims 1-13, or a pharmaceutically acceptable salt or zwitterion thereof, wherein linker is —NH-Q-NH—, —NH—O-Q, or the combination thereof.

15. The compound of any one of claims 1-14, or a pharmaceutically acceptable salt or zwitterion thereof, wherein linker is —NH—CH₂CH₂—NH—, —NH—O—CH₂CH₂—, —NH—O—CH₂C(═O)—, or the combination thereof.

16. The compound of any one of claims 1-15, or a pharmaceutically acceptable salt or zwitterion thereof, wherein R₈ or R₉ is COOH.

17. The compound of any one of claims 1-16, or a pharmaceutically acceptable salt or zwitterion thereof, wherein one of R₈ and R₉ is COOH, and the other one of R₈ and R₉ is —NH—C(═O)—V.

18. The compound of any one of claims 1-17, or a pharmaceutically acceptable salt or zwitterion thereof, wherein W is represented by —C(═O)—U₁-R₈ or —NH—U₁-R₈.

19. The compound of any one of claims 1-18, or a pharmaceutically acceptable salt or zwitterion thereof, wherein
U₁ is -Q-NR₈-Q- and
each Q is independently a —(C₁-C₁₂)alkylene optionally substituted by one or more selected from the group consisting of —OH, —COOH, ═O, and —NH₂.

20. The compound of any one of claims 1-19, or a pharmaceutically acceptable salt or zwitterion thereof, W is Sid and Sid-COOH or Sid-OH is represented by R₈—U₁-R₉, wherein
R₈ is —NH—C(═O)—V,
R₉ is —COOH,
U₁ is -Q-NR₈-Q-
V is 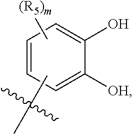
R₈ is Cl, and
m is 0 or 1.

21. The compound of any one of claims 1-16, or a pharmaceutically acceptable salt or zwitterion thereof, W is represented by —C(═O)—U₁-R₈ or —NH—U₁-R₈, wherein
R₈ is —NH—C(═O)—V,
U₁ is -Q-NR₈-Q-
V is 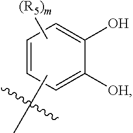
R₅ is Cl, and
m is 0 or 1.

22. The compound of claim 1, wherein Sid is derived from catecholates, hydroxamates, carboxylates, ferrichrome, deferoxamine, desferrioxamine, fusarinine C, ornibactin, rhodotorulic acid, enterobactin, bacillibactin, vibriobactin, azotobactin, pyoverdine, yersiniabactin, aerobactin, simochelin, alcaligin, mycobactin, staphyloferrin A, or petrobactin.

23. The compound of claim 1, wherein Sid is derived from Achromobactin, Acinetobactin, Acinetoferrin, Aerobactin, Aeruginic, Agrobactin, Agrobactin A, Albomycin 271, Alcaligin 230, Alterobactin A, Alterobactin B, Aminochelin 262, Amonabactin P693, Amonabactin P750, Amonabactin T732, Amonabactin T789, Amphibactin B, Amphibactin C, Amphibactin D, Amphibactin E, Amphibactin F, Amphibactin G, Amphibactin H, Amphibactin I, Amycolachrome 235, Anachelin 1, Anachelin 2, Anguibactin 247, Aquachelin A, Aquachelin B, Aquachelin C, 2, Aquachelin D, Arthrobactin, Arthrobactin 199, Asperchrome A, Asperchrome B1, Asperchrome B2, Asperchrome B3, Asperchrome C, Asperchrome D1, Asperchrome D2, Asperchrome D3, Asperchrome E, Asperchrome F1, Asperchrome F2, Asperchrome F3, Aspergillic acid, Avenic acid, Awaitin A, Awaitin B, Awaitin C, Azotobactin 236, Azotobactin D, Azotobactin 87, Azotochelin, Azotochelin 236, Azoverdin 174, Bacillibactin 85, Basidiochrome 46, Biscatechol, Bisucaberin 232, Carboxymycobactin 107, Carboxymycobactin 1, Carboxymycobactin 2, Carboxymycobactin 3, Carboxymycobactin 4, Cepabactin 266, Chrysobactin 261, Citrate 260, Coelichelin 72, 3, Coprogen 51, Coprogen B, Corynebactin 84, Danoxamine, Deoxydistichonic acid, 2′-Deoxymugineic acid, Deoxyschizokinen 251, Des(diserylglycyl)-ferrirhodin 45, Desacetylcoprogen 52, Desferrioxamine A1, Desferrioxamine A2, Desferrioxamine B, Desferrioxamine D1, Desferrioxamine D2, Desferrioxamine E, Desferrioxamine Etl 21A, Desferrioxamine Et2 21B, Desferrioxamine Et3 21C, Desferrioxamine G1, Desferrioxamine G2A, Desferrioxamine G2B, Desferrioxamine G2C, Desferrioxamine H, Desferrioxamine P1, Desferrioxamine T1, Desferrioxamine T2, Desferrioxamine T3, Desferrioxamine T7, Desferrioxamine T8, Desferrioxamine Tel 21D, Desferrioxamine Te2 21E Desferrioxamine Te3 21F, Desferrioxamine X1, Desferrioxamine X2, 4, Desferrioxamine X3, Desferrioxamine X4, Desferrithiocin, Diamine biscatechol, Dihydrobenzolate, 2,3-Dihydroxybenzoylserine, Dimerum acid, Dimethylcoprogen, Dimethylneocoprogen I, Dimethyltriornicin, Distichonic acid, Enantio Rhizoferrin, Enantio-Pyochelin, Enterobactin, Enterochelin, Exochelin MN, Exochelin MS, Ferrichrome, Ferrichrome A, Ferrichrome C, Ferrichrysin, Ferricrocin, Ferrioxamine, Ferrimycin A, Ferrirhodin, Ferrirubin, Ferrocin A, Fimsbactin A, Fluvibactin, Formobactin, Foroxymithine, Fusarinine A, Fusarinine B, Fusarinine C, Heterobactin A, Heterobactin B, Hydroxycopropen, Hydroxypyridone, Hydroxyisoneocoprogen I, 3-Hydroxymugineic acid, 5, Hydroxy-neocoprogen I, Isoneocoprogen I, Isopyoverdin BTP1, Isopyoverdin 6.7, Isopyoverdin 7.13, Isopyoverdin 90-33, Isopyoverdin 90-44, Isopyoverdin 10.7, Isotriornicin, Itoic acid, Loihichelin A, Loihichelin B, Loihichelin C, Loihichelin D, Loihichelin E, Loihichelin F, Maduraferrin, Malonichrome, Marinobactin A, Marinobactin B, Marinobactin C, Marinobactin D1, Marinobactin D2, Marinobactin E, Micacocidin, Mugineic acid, Mycobactin, Mycobactin A, Mycobactin Av, Mycobactin F, Mycobactin H, Mycobactin J, Mycobactin M, Mycobactin N, 6, Mycobactin NA, Mycobactin P, Mycobactin R, Mycobactin S, Mycobactin T, Myxochelin, ω-N-acetyl-ω-N-hydroxyl-α-aminoalkane, ε-N-acetyl-ε-N-hydroxyl L-lysine, δ-N-acetyl-δ-N-hydroxyl L-ornithine, Nannochelin A, Nannochelin B, Nannochelin C, Neocoprogen I, Neocoprogen II, Neurosporin, Nocobactin, Nocobactin NA, Ochrobactin A, Ochrobactin B, Ochrobactin C, Ornibactin-C4, Ornibactin-C6, Ornibactin-C8, Ornicorrugatin, palmitoylcoprogen, Parabactin, Parabactin A, Petrobactin, Petrobactin disulphonate, Petrobactin sulphonate, Pistillarin, Polyamine biscatechol, Protochelin, Pseudoalterobactin A, Pseudoalterobactin B, Pseudobactin 112, Pseudobactin 589A, 7, Putrebactin, Pyochelin, Pyoverdin A214, Pyoverdin BTP2, Pyoverdin C, Pyoverdin CHAO, Pyoverdin D-TR133, Pyoverdin E, Pyoverdin G R Pyoverdin GM, Pyoverdin I-III, Pyoverdin P19, Pyoverdin Pau, Pyoverdin PL8, Pyoverdin PVD, Pyoverdin R′, Pyoverdin Thai, Pyoverdin TII, Pyoverdin 1, Pyoverdin 11370, Pyoverdin 13525, Pyoverdin 1547, Pyoverdin 17400, Pyoverdin 18-1, Pyoverdin 19310, Pyoverdin 2192, Pyoverdin 2392, Pyoverdin 2461, Pyoverdin 2798, Pyoverdin 51W, Pyoverdin 9AW, Pyoverdin 90-51, Pyoverdin 95-275, Pyoverdin 96-312, Pyoverdin 96-318, Pyoverdin, Pyoverdin 6.1, Pyoverdin 6.2, Pyoverdin 6.3, Pyoverdin 6.4, Pyoverdin 6.5, Pyoverdin 6.6, Pyoverdin 6.8, Pyoverdin 7.1, Pyoverdin 7.2, Pyoverdin 7.3, Pyoverdin 7.4, Pyoverdin 7.5, Pyoverdin 7.6, Pyoverdin 7.7, Pyoverdin 7.8, Pyoverdin 7.9, Pyoverdin 7.10, Pyoverdin 7.11, Pyoverdin 7.12, Pyoverdin 7.14, Pyoverdin 7.15, Pyoverdin 7.16, Pyoverdin 7.17, Pyoverdin 7.18, Pyoverdin 7.19, Pyoverdin 8.1, Pyoverdin 8.2, Pyoverdin 8.3, Pyoverdin 8.4, Pyoverdin 8.5, Pyoverdin 8.6, Pyoverdin 8.7, Pyoverdin 8.8, Pyoverdin 8.9, Pyoverdin 9.1, Pyoverdin 9.2, Pyoverdin 9.3, Pyoverdin 9.4, Pyoverdin 9.5, Pyoverdin 9.6, Pyoverdin 9.7, Pyoverdin 9.8, Pyoverdin 9.9, Pyoverdin 9.10, Pyoverdin 9.11, Pyoverdin 9.12, Pyoverdin 10.1, Pyoverdin 10.2, Pyoverdin 10.3, Pyoverdin 10.4, Pyoverdin 10.5, Pyoverdin 10.6, Pyoverdin 10.8, Pyoverdin 10.9, Pyoverdin 10.10, Pyoverdin 11.1, Pyoverdin 11.2, Pyoverdin 12, Pyoverdin 12.1, Pyoverdin 12.2, Pyoverdine, Pyridoxatin, Quinolobactin, Rhizobactin, 10, Rhizobactin, Rhizoferrin, Rhizoferrin analogues 88A-88E, Rhodotrulic acid, Salmochelin S1, Salmochelin S2, Salmochelin S4, Salmochelin SX, Salmycin A, Schizokinen, Serratiochelin, Siderochelin A, Snychobactin A, Snychobactin B, Snychobactin C, Staphyloferrin A, Staphyloferrin B, Tetraglycine ferrichrome, Thiazostatin, Triacetylfusarinine, Tricatechol, Triornicin, Vibriobactin, Vibrioferrin, Vicibactin, Vulnibactin, or Yersiniabactin.

24. The compound of any one of claims 1-23, or a pharmaceutically acceptable salt or zwitterion thereof, wherein Sid is derived from Aerobactin, Agrobactin, Arthrobactin, Awaitin A, Awaitin B, Awaitin C, Azotochelin, Biscatechol, Danoxamine, Dihydrobenzolate, Enterobactin, Ferricrocin, Ferrioxamine, Fimsbactin A, Foroxymithine, Hydroxypyridone, Mycobactin, ω-N-acetyl-ω-N-hydroxyl-α-aminoalkane, ε-N-acetyl-ε-N-hydroxyl L-lysine, δ-N-acetyl-δ-N-hydroxyl L-ornithine, Parabactin, Pyoverdine, Rhodotrulic acid, Schizokinen, or Tricatechol.

25. The compound of any one of claims 1-24, or a pharmaceutically acceptable salt or zwitterion thereof, wherein the compound is selected from the group consisting of:

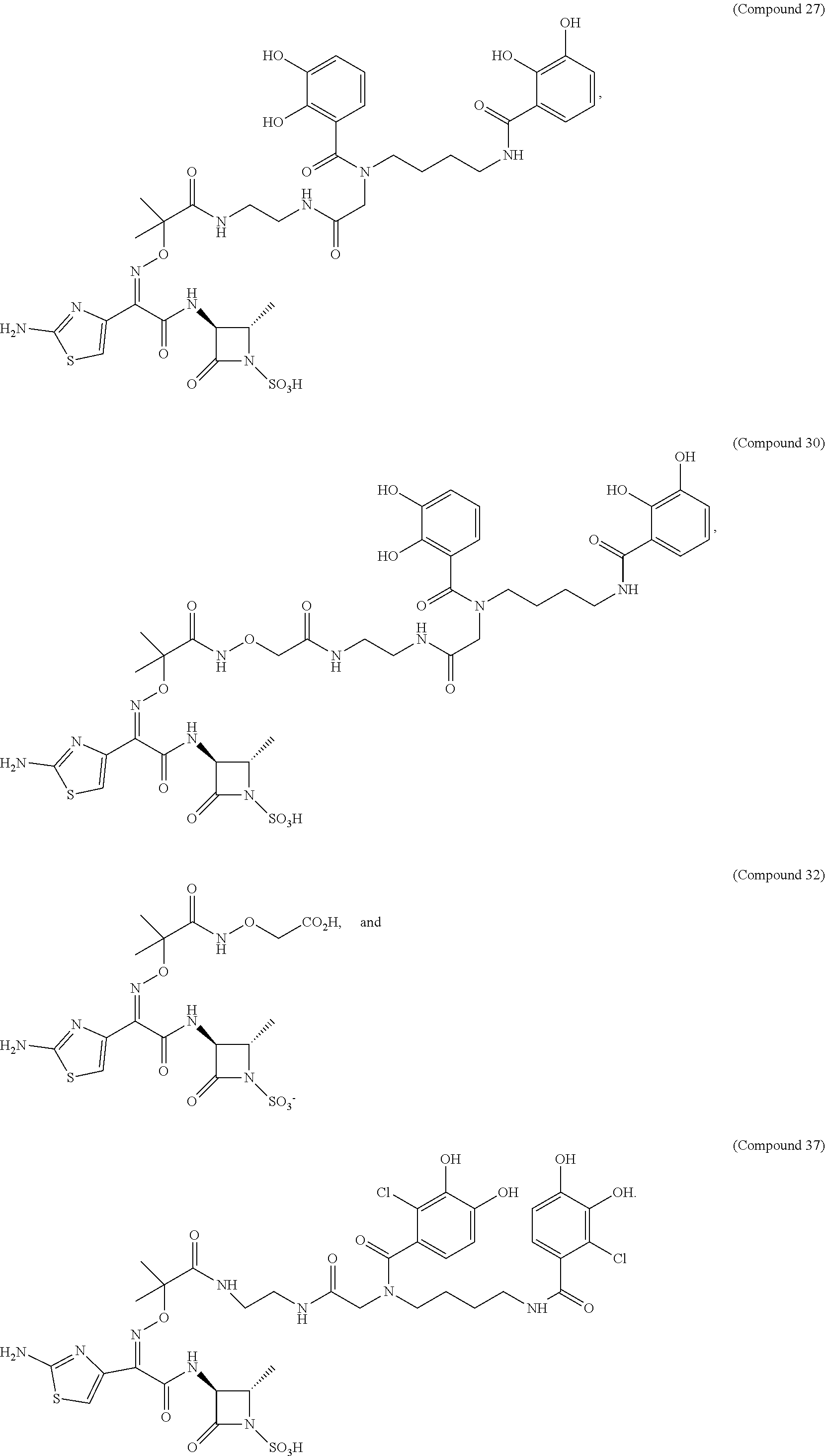

26. A pharmaceutical composition comprising the compound of any one of claims 1-25, or a pharmaceutically acceptable salt or zwitterion thereof, and at least one pharmaceutically acceptable carrier, diluent, or excipient.

27. A method of treating a bacterial infection in a subject in need thereof, comprising administering to the said subject a therapeutically effective amount of the compound according to any one of claims 1-25, or a pharmaceutically acceptable salt or zwitterion thereof or a therapeutically effective amount of the pharmaceutical composition of claim 26.

28. The method of claim 27, wherein the bacterial infection is a gram-negative bacterial infection.

29. The method of claim 27, wherein the bacterial infection is a gram-positive bacterial infection.

30. The method of any one of claims 27-29, wherein the method comprises administering (3-lactams.

31. The method of any one of claims 27-30, wherein the bacterial infection is caused by Pseudomonas aeruginosa, Acinetobacter baumannii, Escherichia coli, and Klebsiella pneumonia.

32. The method of any one of claims 27-31, wherein the subject is a human subject.

33. The method of any one of claims 27-31, wherein the subject is an animal subject.

34. The method of any one of claims of claims 27-33, further comprising administering an effective amount of an additional antibiotic agent.

35. The method of claim 34, wherein the additional antibiotic compound is selected from the group consisting of penicillin, methicillin, oxacillin, nafcillin, cloxacillin, dicloxacillin, flucloxacillin, temocillin, amoxicillin, ampicillin, co-amoxiclav, azlocillin, carbenicillin, ticarcillin, mezlocillin, piperacillin, cephalexin, cephalothin, CXA-101, cefazolin, cefaclor, cefuroxime, cefamandole, cefotetan, cefoxitin, ceftriaxone, cefotaxime, cefpodoxime, cefixime, ceftazidime, ceftobiprole medocaril, cefepime, cefpirome, ceftaroline, imipenem, meropenem, ertapenem, faropenem, sulopenem, doripenem, PZ-601 (Protez Pharmaceuticals), ME1036 (Forest Labs), BAL30072, MC-1, tomopenem, tebipenemn, aztreonam, tigemonam, nocardicin A, and tabtoxinine-β-lactam.

36. The method of any one of claims 27-35, wherein the bacterial infection is resistant to one or more antibiotics.

37. The method of any one of claims 27-36, wherein the bacterial infection causes a disease selected from the group consisting of urinary tract infections, pneumonia, prostatitis, skin and soft tissue infections, sepsis, and intra-abdominal infections.

38. A process of preparing a compound of Formula (I), or a pharmaceutically acceptable salt or zwitterion thereof, the process comprising: contacting a compound of Formula (I′-1) 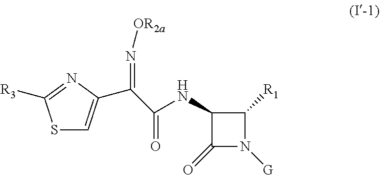
with a compound of Formula (II′-1) or Formula (II′-2)
R₈—U₁—NH₂, (II′-1) or
R₈—U₁-L′—NH₂ (II′-2)
under suitable conditions to produce the compound having Formula (I) 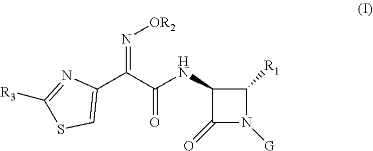
wherein
G is —OR^{A}, —OCH₂C(═O)X, OCHRC(═O)X, —C(═O)X, —S(═O)₂X, —OS(═O)₂X, —C(═O)NH—S(═O)₂G′, P(═O)X or —P(═O)X,
G′ is OR^{A}, —OCH₂C(═O)X, OCHRC(═O)X, —C(═O)X, —S(═O)₂X, —OS(═O)₂X, P(═O)X or —P(═O)X,
X is H, —OR^{A} or —N(R^{A})₂,
R₁ is H, or —(C₁-C₁₂)alkyl optionally substituted by X or Z,
Z is R^{A}, OR^{A}, —SR^{A}, —C(═O)N(R^{A})₂, or —N(R^{A})₂,
R₂ₐ is —CH₂C(═O)OH, —CHR⁴C(═O)OH, —C(R⁴)₂C(═O)OH, or —C(R⁴)₂C(═O)NHOCH₂C(═O)OH, —C(R⁴)₂C(═O)NHOC(R⁴)₂C(═O)OH,
R₂ is —CH₂C(═O)W′, —CHR⁴C(═O)W′, —C(R⁴)₂C(═O)W′, —C(R⁴)₂C(═O)NHOCH₂C(═O)W′, —C(R⁴)₂C(═O)NHOCH₂C(═O)OH, —C(R⁴)₂C(═O)NHOC(R⁴)₂C(═O)OH, or —C(R⁴)₂C(═O)NHOC(R⁴)₂C(═O)W′,
R₃ is H, Z, or R⁴,
each R⁴ is independently H, Z, —(C₁-C₁₂)alkyl, —(C₃-C₈)cycloalkyl, —(C₃-C₆)heterocycloalkyl, or —(C₁-C₈)N(R^{A})R^{B}, or two R⁴ are taken together with the carbon atom to which both are attached to form a —(C₃-C₅)cycloalkyl or —(C₃-C₆)heterocycloalkyl,
each R^{A} is independently H, —(C₁-C₁₂)alkyl, —(C₃-C₅)cycloalkyl, phenyl, aryl, or heteroaryl,
R^{B} is H, —(C₁-C₁₂)alkyl, —(C₃-C₈)cycloalkyl, or phenyl,
W′ is —NH-U₁-R₈ or —NH-L′—U₁-R₈,
L′ is -Q-C(═O)O—, —C(═O)-Q-C(═O)O—, —C(═O)-Q-, —C(═O)O-Q-, —C(═O)-Q-C(═O)—, —O-Q-, -Q-, -Q-NH—, -Q-O—, or the combination thereof,
each Q is independently a —(C₁-C₁₂)alkylene optionally substituted by one or more selected from the group consisting of —OH, —COOH, ═O, —NH₂, —(C₁-C₁₂)alkyl, —(C₁-C₁₂)alkenyl, —(C₁-C₁₂)alkylene —OH, —(C₁-C₁₂)alkylene —NH₂, —NH—(C₁-C₁₂)alkylene-COOH, —NH—C(═O)—(C₁-C₁₂)alkylene —COOH, —C(═O)—(C₁-C₁₂)alkylene —C(═O)—NH₂, —O—(C₁-C₁₂)alkylene-OH, —NH—(C₁-C₁₂)alkylene —NH₂, and —NH—(C₁-C₁₂)alkylene —OH.
U₁ is a covalent bond, or optionally substituted -Q-, —C(═O)—, —O—C(═O)—, —CHR₈—, —CR₁₀R₈—, —C(═O)—NH—, —C(═O)—NR₈—, —C(═O)—N(OH)—, —NH—, —NR₈—, —NR₁₀—, —NH—CHR₈—C(═O)—, —R₁₁—, or the combination thereof,
each R₈ is each independently H, —OH, —COOH, —NH₂, -Q-OH, —V, -Q-V, —NH—C(═O)—V, -Q-NH—C(═O)—V, —NR₁₀—C(═O)—V, —NR₁₀—V, —C(═O)—V, —O—C(═O)—V, —C(═O)—R₁₁—V, —NH—C(═O)—R₁₁—V, —C(═O)—R₁₁-aryl, or L′-H,
R₁₀ is —NH—C(O)-Q-COOH, —C(O)-Q-COOH, -Q-COOH, or —COOH,
R₁₁ is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S,
V═—N(OH)—C(═O)—NH₂, —N(OH)—C(═O)H, N(OH)—C(═O)-L′, —N(OH)—C(═O)—(C₁-C₁₂)alkyl, —N(OH)—C(═O)—(C₁-C₁₂)alkenyl, —N(OH)—C(═O)-Q-OH, —N(OH)—C(═O)-Q-COOH, 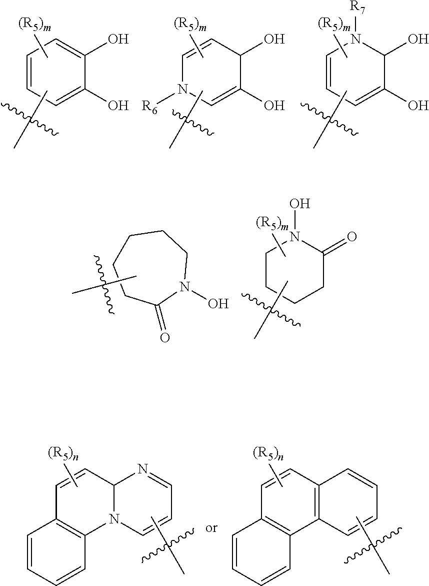
m is an integral selected from 0-3,
n is an integral selected from 0-10,
each R₅ is independently —(C₁-C₁₂)alkyl, halogen, —OH, —COOH, —NH₂, -L′-H, or —C(═O)-L′-H, and
R₆ and R₇ are independently H, —COOH, or —OR^{A}.

39. The process of claim 38, where the process comprises coupling the compound of (I′-1) with the compound of Formula (II′-1) or Formula (II′-2) to produce the compound of Formula (I).

40. The process of any one of claims 38-39, the coupling is at room temperature.

41. The process of any one of claims 38-40, the coupling comprises contacting the compound of (I′-1) and the compound of Formula (II′-1) or Formula (II′-2) with a coupling reagent.

42. The process of any one of claims 38-41, the process comprises the steps of:
(a) mixing the compound of Formula (I′-1) in a solvent with a coupling reagent,
(b) stirring to obtain a solution,
(c) mixing the compound of Formula (II′-1) or (II′-2) in a solvent with the solution of step (b), optionally further mixing with an additional coupling reagent,
(d) stirring, and
(e) obtaining the compound of Formula (I).

43. The process of claim 42, wherein the stirring at step (b) or step (d) is at room temperature.

44. The process of any one of claims 42-43, wherein step (e) comprises reduced pressure evaporation.

45. The process of any one of claims 42-44, the solvent in step (a) or step (c) is water, tetrahydrofuran (THF), dimethylformamide (DMF), or a mixture thereof.

46. The process of claim 38, wherein the process comprises the steps of:
(a) mixing the compound of Formula (I′-1) and the compound of Formula (II′-1) or Formula (II′-2) in a solvent to from a solution,
(b) maintaining or adjusting the pH of the solution,
(c) dissolving a coupling reagent in a solvent, and
(d) mixing the solution of step (c) with the solution of step (b) while maintaining or adjusting the pH of the solution, and
(e) obtaining the compound of Formula (I).

47. The process of claim 46, wherein the pH of the solution in step (b) or step (d) is about 4.5.

48. The process of any one of claims 38-47, wherein the coupling reagent is HBTU, DIPEA, N-hydroxysuccinimide, EDC-HCl, or mixture thereof.

49. The process of any one of claims 38-48, wherein the solvent in step (a) or step (c) is water, tetrahydrofuran (THF), dimethylformamide (DMF), or a mixture thereof.

50. The process of any one of claims 38-49, wherein G is S(═O)₂OH.

51. The process of any one of claims 38-50, wherein R₁ is —(C₁-C₁₂)alkyl.

52. The process of any one of claims 38-51, wherein R₁ is —CH₃.

53. The process of any one of claims 38-52, R⁴ is —CH₃.

54. The process of any one of claims 38-53, wherein R₃ is —NH₂.

55. The process of any one of claims 38-54, wherein V is

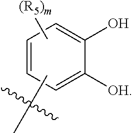

56. The process of any one of claims 38-55, wherein Formula (I′-1) is represented by Formula (I′-2)

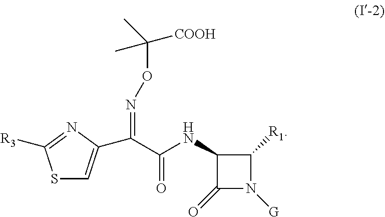

57. The process of any one of claims 38-56, wherein Formula (I′-1) is represented by Formula (I′-3)

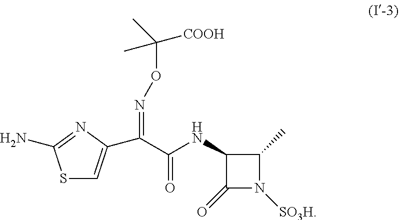 [SMILES: C[C@H]1[C@H](NC(=O)/C(=N\OC(C)(C)C(=O)O)c2csc(N)n2)C(=O)N1S(=O)(=O)O]

58. The process of any one of claims 38-57, wherein L′—NH₂ is —NH-Q-NH₂ or -Q-O—NH₂, or the combination thereof.

59. The process of any one of claims 38-58, wherein L′—NH₂ is —NH—CH₂CH₂—NH₂, —CH₂CH₂—ONH₂, —C(═O)CH₂—ONH₂, or the combination thereof.

60. The process of any one of claims 38-59, wherein R₈ is —NH—C(═O)—V.

61. The process of any one of claims 38-60, wherein
U₁ is -Q-NR₈-Q- and
each Q is independently a —(C₁-C₁₂)alkylene optionally substituted by one or more selected from the group consisting of —OH, —COOH, ═O, and —NH₂.

62. The process of any one of claims 38-61, wherein
R₈ is —NH—C(═O)—V,
U₁ is -Q-NR₈-Q-
V is 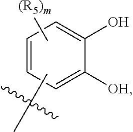
R₅ is Cl, and
m is 0 or 1.

63. The process of any one of claims 38-62 wherein Formula (II′-1) or Formula (II′-2) is selected from the group consisting of 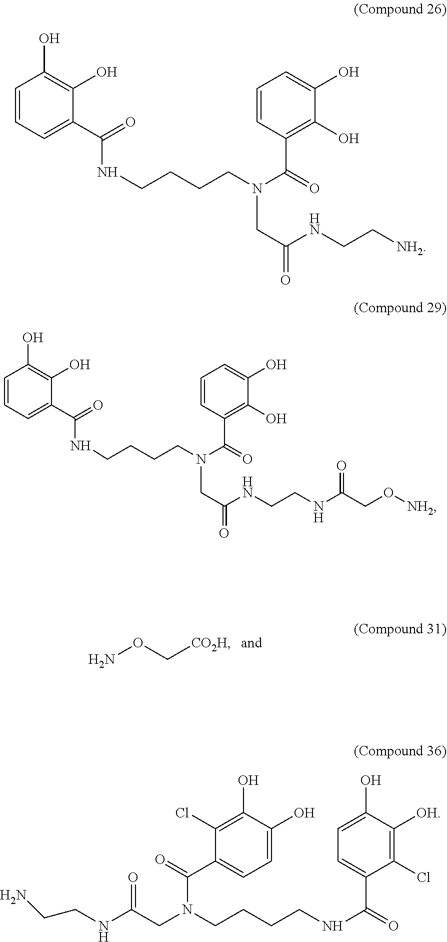 [SMILES: NCCNC(=O)CN(CCCCNC(=O)c1cccc(O)c1O)C(=O)c1cccc(O)c1O ; NOCC(=O)NCCNC(=O)CN(CCCCNC(=O)c1cccc(O)c1O)C(=O)c1cccc(O)c1O ; NOCC(=O)O ; "and" ; NCCNC(=O)CN(CCCCNC(=O)c1ccc(O)c(O)c1Cl)C(=O)c1ccc(O)c(O)c1Cl]
